(19) United States
(12) Patent Application Publication
(10) Pub. No.: US 2008/0194536 A1
(43) Pub. Date: Aug. 14, 2008

(54) Cyanoarylamines

(76) Inventors: Marlys Hammond, King of Prussia, PA (US); David G. Jones, Research Triangle Park, NC (US); Istvan Kaldor, Research Triangle Park, NC (US); Lara S. Kallander, King of Prussia, PA (US); Xi Lang, Research Triangle Park, NC (US); Scott Kevin Thompson, King of Prussia, PA (US); Philip Stewart Turnbull, Research Triangle Park, NC (US); David G. Washburn, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/911,537

(22) PCT Filed: Apr. 14, 2006
(86) PCT No.: PCT/US06/14286

§ 371 Date: Oct. 15, 2007

Related U.S. Application Data
(60) Provisional application No. 60/671,592, filed on Apr. 15, 2005.

(51) Int. Cl. A61K 31/397; C07C 237/02; A61K 31/16; C07D 213/02; A61K 31/44; C07D 307/02; A61K 31/34; A61K 31/54; A61K 31/40; A61P 15/00; C07D 207/00; A61K 31/5375; C07D 333/02; A61K 31/381; C07D 295/00; A61K 31/445; C07D 205/04

(52) U.S. Cl. 514/210.17; 564/163; 514/619; 546/336; 514/357; 549/496; 514/471; 549/76; 514/438; 546/226; 514/330; 548/953; 544/162; 514/237.5; 544/59; 514/227.5; 548/530; 514/423

(57) ABSTRACT

The present invention relates to a compound represented by the following formula:

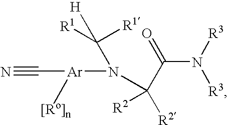

(I) or a pharmaceutically acceptable salt thereof, or a pharmaceutically acceptable solvate thereof, or a combination thereof, wherein R^{o}, R¹, R^{1′}, R², R^{2′}, R³ and n are defined herein. The present invention further relates to a method of treating a patient from endometreosis or uterine fibroids.

### BACKGROUND OF THE INVENTION

The present invention relates to a cyanoarylamine that is useful as a progesterone receptor modulator.

Endometriosis is a disease characterized by the growth of endometrial tissue (called lesions) at extrauterine sites. This lesion attachment can result in pain, dysmenorrhea, dyspareunia, and infertility. It is estimated that greater than 80% of patients presenting with chronic pelvic pain are eventually diagnosed with endometriosis. The prevalence of the disease is about 7-10% of women of reproductive years with a familial association risk increase of 10-fold. Definitive diagnosis is only reached by laparoscopy, but typically there is about a ten year delay from disease onset to conclusive diagnosis. Consistent with their uterine origins, it is believed that the endometriotic lesions are hormonally dependent upon estrogen; consequently, therapies that functionally antagonize estrogen production or action, such as drugs containing progesterone receptor (PR) modulators, are efficacious in alleviating symptoms. Current therapeutic goals include reducing pain with anti-inflammatory agents and suspending the ovarian cycle using hormonal modulation drugs.

Another disease believed to be hormonally responsive to estrogen is uterine leiomyomas (fibroids), which appear as benign uterine smooth muscle tumors occurring primarily in women of reproductive age. Fibroids occur at rates of 20-25% and are the leading indication for hysterectomies. The most common symptoms are menorrhagia, pelvic pain/discomfort, bladder and bowel compression symptoms, and possibly infertility. Medical treatments for leiomyomas consist of those commonly prescribed for endometriosis, with treatments containing progesterone receptor modulators being most common due to safety, tolerability, ease of use and cost.

Most drug development has focused on modulation by full agonism or antagonism of progesterone receptors. For example, progestins are molecules that interact with progesterone receptor to activate or repress gene expression in target cells in a manner presumed to be progesterone-like. Though progestins are used in oral contraception, hormone therapy, and treatment of reproductive disorders, such as endometriosis and leiomyomas, these agents cause a number of adverse effects, including breakthrough bleeding, mood altering, acne, weight gain, and breast tenderness. Paradoxically, progesterone receptor antagonists such as mifepristone have been suggested as potential therapies, but the data are limited with few patients and no placebo-controlled randomized trials.

D. DeManno et al. (Steroids 68 (2003) 1019-1032), report that asoprisnil is a progesterone receptor modulator with mixed agonist/antagonistic activities. While the efficacy of the agent in treatment of endometriosis or fibroids is uncertain, early data from healthy female subjects indicate that the agent induces endometrial atrophy and amenorrhea, which suggests a predominantly progesterone receptor antagonist action in humans. Unfortunately, PR antagonists such as RU-486 tend to be abortifacient.

Accordingly, it would be desirable to discover a way to suppress estrogen-dependent endometriotic growth while reducing the systemic effects associated with current progesterone receptor modulating therapy.

### SUMMARY OF THE INVENTION

In a first aspect, the present invention provides a A compound represented by the following formula:

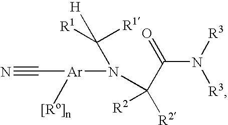

or a pharmaceutically acceptable salt thereof, or a pharmaceutically acceptable solvate thereof, or a combination thereof;

wherein n is 0, 1, or 2;

Ar is phenyl or naphthyl;

each R^{o} is independently CF₃, halo, C₁₋₆-alkyl, CN, or substituted C₁₋₆-alkyl;

R¹ and R^{1′} are each independently H, C₁₋₆-alkyl, substituted C₁₋₆-alkyl, C₂₋₆-alkenyl, C₃₋₆-cycloalkyl, substituted C₃₋₆-cycloalkyl, aryl, substituted aryl, heteroaryl, or substituted heteroaryl, or together with the carbon atom to which they are both attached form a C₅₋₆-cycloalkyl or C₅₋₆-cycloalkenyl group;

R² and R^{2′} are each independently H, C₁₋₆-alkyl, substituted C₁₋₆-alkyl, C₃₋₆-cycloalkyl, substituted C₃₋₆-cycloalkyl, —(CH₂)ₘ—X—C₃₋₆-cycloalkyl, —(CH₂)ₘ—X-substituted C₃₋₆-cycloalkyl, —(CH₂ₘ—X-phenyl, —(CH₂)ₘ—X-substituted phenyl, —(CH₂)ₘ—X-pyridyl, or —(CH₂)ₘ—X-substituted pyridyl; and X is a bond, —O— or —S—, where m is 0-4 when X is a bond and m is 1-4 when X is —O— or —S—; with the proviso that R² and R^{2′} are not both H; and

each R³ is independently C₁₋₆-alkyl, substituted C₁₋₆-alkyl, C₁₋₆-alkenyl, or propargyl, or together with the nitrogen to which they are attached form a 3-6-membered heterocycloalkyl group;

with the proviso that when a) Ar is phenyl; b) R² is methyl or ethyl; c) R^{2′} is H; d) each R³ is methyl; e) R¹ is H; f) R^{o} is CF₃ ortho to the CN, and g) n=1; then R^{1′} is not cyclopropyl or CF₃.

In a second aspect, the present invention further relates to a method of treating a patient comprising administering to the patient an effective amount of a compound of the formula or a pharmaceutically-acceptable salt thereof, or a solvate thereof or combination thereof to treat endometreosis or uterine fibroids.

In a third aspect, the present invention relates to a composition comprising the compound of the formula of the present invention and a pharmaceutically acceptable carrier therefore.

Compounds of the present invention are useful as progesterone receptor modulators.

### DETAILED DESCRIPTION OF THE INVENTION

The present invention is a compound represented by the following formula:

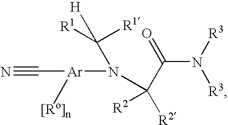

or a pharmaceutically acceptable salt thereof, or a pharmaceutically acceptable solvate thereof, or a combination thereof;

wherein n is 0, 1, or 2;

Ar is phenyl or naphthyl;

each R^{o} is independently CF₃, halo, C₁₋₆-alkyl, CN, or substituted C₁₋₆-alkyl;

R¹ and R^{1′} are each independently H, C₁₋₆-alkyl, substituted C₁₋₆-alkyl, C₂₋₆-alkenyl, C₃₋₆-cycloalkyl, C₃₋₆-substituted cycloalkyl, aryl, substituted aryl, heteroaryl, or substituted heteroaryl, or together with the carbon atom to which they are both attached form a C₅₋₆-cycloalkyl or C₅₋₆-cycloalkenyl group;

R² and R^{2′} are each independently H, C₁₋₆-alkyl, substituted C₁₋₆-alkyl, C₃₋₆-cycloalkyl, substituted C₃₋₆-cycloalkyl, —(CH₂)ₘ—X—C₃₋₆-cycloalkyl, —(CH₂)ₘ—X-substituted C₃₋₆-cycloalkyl, —(CH₂)ₘ—X-phenyl, —(CH₂)ₘ—X-substituted phenyl, —(CH₂)ₘ—X-pyridyl, or —(CH₂)ₘ—X-substituted pyridyl; and X is a bond, —O— or —S—, where m is 0-4 when X is a bond and m is 1-4 when X is —O— or —S—; with the proviso that R² and R^{2′} are not both H; and

each R³ is independently C₁₋₆-alkyl, substituted C₁₋₆-alkyl, C₁₋₆-alkenyl, or propargyl, or together with the nitrogen to which they are attached form a 3-6-membered heterocycloalkyl group;

with the proviso that when a) Ar is phenyl; b) R² is methyl or ethyl; c) R^{2′} is H; d) each R³ is methyl; e) R¹ is H; f) R^{o} is CF₃ ortho to the CN, and g) n=1; then R^{1′} is not cyclopropyl or CF₃.

The cyanoarylamine of the present invention is a 1,4-cyanoarylamine, that is, the partial structure NC—Ar—N< refers to a 1,4-relationship between the cyano group and the nitrogen atom. Thus, where Ar is phenyl, the cyano group and nitrogen atom are para to each other.

As used herein, halo refers to —F, —Cl, —Br, or —I; C₁₋₆-alkyl refers to a straight or branched chain monovalent radical of 1 to 6 carbon atoms, including, methyl, ethyl, n-propyl, isopropyl, n-butyl, isobutyl, t-butyl, n-pentyl, isopentyl, neopentyl, and n-hexyl and isomers thereof.

Substituted C₁₋₆-alkyl refers to alkyl groups substituted with up to three atoms or groups selected from —F, —Cl, —Br, —OH, —SH, —COOH, —N(R⁴)₂ and —CN, where each R⁴ is independently H or C₁₋₆-alkyl. Substituted C₁₋₆-alkyl also includes incorporated atoms or groups selected from —O—, —S—, and —NR⁴—. Examples of suitable substituted alkyl groups include but are not limited to —CF₃, —CH₂CF₃, and —CH(OCH₃)₂.

Substituted C₃₋₆-cycloalkyl refers to cycloalkyl groups substituted with up to three atoms or groups selected from F, —Cl, —Br, —OH, —SH, —COOH, —N(R⁴)₂ and —CN, where R⁴ is previously defined.

Examples of C₂₋₆-alkenyl groups include vinyl, allyl, and isopropenyl groups. Examples of suitable aryl groups include phenyl and naphthyl groups; suitable heteroaryl groups include pyridyl, furyl, and thienyl groups. Substituted aryl (including phenyl) and substituted heteroaryl (including pyridinyl) refer to aryl and heteroaryl groups respectively substituted with up to three atoms or groups selected from —F, —Cl, —Br, —CF₃, —CH₃, —CH₂CH₃, —OH, —OCH₃, —SH, CN, —COOH, and —(CH₂)ₚN(R⁴)₂ groups, where p is 0, 1, or 2.

Examples of 3-6-membered heterocycloalkyl groups include aziridinyl, azetidinyl, pyrrolidinyl, piperidinyl, piperazinyl, thiazolidinyl, thiomorpholino, and morpholino groups.

The term “IC₅₀” is used herein to refer to the molar concentration of a compound required to inhibit binding of 50% of Fluormone PL Red to the progesterone receptor. Furthermore, pIC₅₀ is the negative log of the molar IC₅₀.

Pharmaceutically acceptable salts of the compounds of the present invention include salts formed by the addition of an inorganic acid such as hydrochloric acid, hydrobromic acid, sulfuric acid, nitric acid, or phosphoric acid; or by the addition of an organic acid such as acetic acid, fumaric acid, succinic acid, maleic acid, citric acid, benzoic acid, p-toluenesulfonic acid, methanesulfonic acid, naphthalenesulfonic acid, or tartaric acid.

Some of the compounds of the present invention may exist as optical isomers including diastereoisomers and enantiomers, and mixtures of isomers in all ratios including racemic mixtures. The different isomeric forms may be separated or resolved by conventional methods, or any given isomer may be obtained by conventional synthetic methods or by stereospecific or asymmetric synthesis.

The present invention also relates to a pharmaceutical composition comprising the compound of the formula of the present invention and a pharmaceutically acceptable carrier therefor. The composition may be formulated for administration by any route, such as oral, topical or parenteral. The compositions may be in the form of tablets, capsules, powders, granules, lozenges, creams or liquid preparations, such as oral or sterile parenteral solutions or suspensions.

Biological Assays

Abbreviations

Acquest/Biosystems is a multi-mode reader (FP reader); CHAPS refers to 3-cholamidopropyl-dimethylammoniol-propanesulfonate; DTT refers to dithiothreitol.

PR Binding Assay—The assay was performed according to the manufacturers protocol (PR Competitor Assay Kit, Red—(Invitrogen—Product No. P2962)) with minor amendments. Briefly, 40 nM PR-Ligand Binding Domain, 2 nM Fluormone PL Red and 1 mM DTT were dissolved and mixed in Complete PR RED Buffer supplemented with 2 mM CHAPS. 10 μL of the mix was dispensed to each well of Greiner low volume plates, containing compounds at the required concentration. The plates were spun for 1 min at 200 g, covered to protect the reagents from light, and then incubated at room temperature for approximately 2 hours. Plates were read on an Acquest using a 530-25 nm excitation and 580-10 nm emission interference filter and a 561 nm Dichroic mirror.

Data Analysis

All data was normalized to the mean of 16 high and 16 low control wells on each plate. A four parameter curve fit of the following form was then applied

$$y = \frac{a - d}{1 + {( \frac{x}{c} )}^{b}} + d$$

Where a is the minimum, b is the Hill slope, c is the XC50 and d is the maximum. Data is presented as the mean pIC₅₀ with the standard deviation of the mean of n experiments.

Methods of Use

The compounds of the present invention are useful as modulators of progesterone receptors and may be useful in the treatment of disease associated with endometreosis and uterine fibroids. Thus, the present invention further relates to a method of treating a patient comprising administering to the patient an effective amount of a compound of formula I or a pharmaceutically-acceptable salt thereof, or a solvate thereof or combination thereof to treat endometreosis or uterine fibroids.

### EXAMPLES

The following Examples are for illustrative purposes only and are not intended to limit the scope of the invention. The compounds from these Examples exhibit a pIC₅₀ of greater than 5 (i.e., an IC₅₀ of less than 10 μM).

The names and structures of the compounds prepared in the Examples are illustrated in the following Table.

| | | |
|---|---|---|
| Example 1 | N²-[(2-chlorophenyl)methyl]-N²-[4-cyano-3-(trifluoromethyl)phenyl]-N¹,N¹-dimethyl-L-alaninamide | 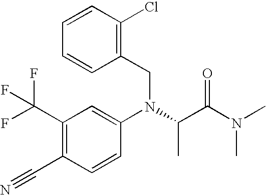 |
| Example 2 | N²-[4-cyano-3-(trifluoromethyl)phenyl]-N²-isobutyl-N¹,N¹-dimethyl-L-alaninamide | 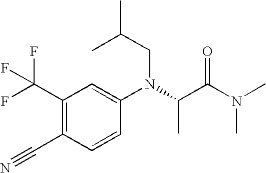 |
| Example 3 | N²-[4-cyano-3-(trifluoromethyl)phenyl]-N¹,N¹-dimethyl-N²-(1-phenylethyl)-L-alaninamide | 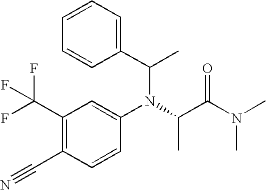 |
| Example 4 | N²-[4-cyano-3-(trifluoromethyl)phenyl]-N¹,N¹-dimethyl-N²-(2-methyl-2-propen-1-yl)-L-isoleucinamide | 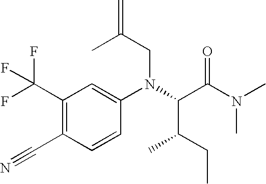 |
| Example 5 | N²-[4-cyano-3-(trifluoromethyl)phenyl]-N¹,N¹-dimethyl-N²-(1-methylethyl)-L-alaninamide | 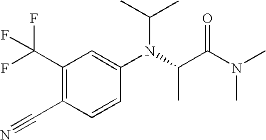 |
| Example 6 | N²-[(2-chlorophenyl)methyl]-N²-[4-cyano-3-(trifluoromethyl)phenyl]-N¹,N¹-dimethyl-L-isoleucinamide | 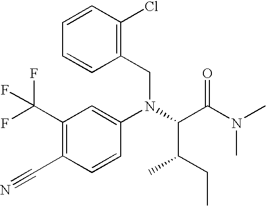 |
| Example 7 | N²-[4-cyano-3-(trifluoromethyl)phenyl]-N¹,N¹-dimethyl-N²-(2-methylpropyl)-L-isoleucinamide | 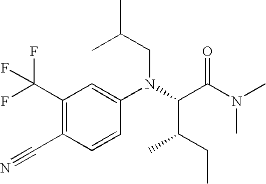 |
| Example 8 | N²-[(2-chlorophenyl)methyl]-N²-[4-cyano-3-(trifluoromethyl)phenyl]-N¹,N¹-dimethyl-L-valinamide | 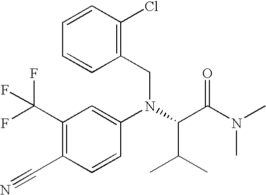 |
| Example 9 | N²-[4-cyano-3-(trifluoromethyl)phenyl]-N¹,N¹-dimethyl-N²-{[2-(trifluoromethyl)phenyl]methyl}-L-valinamide | 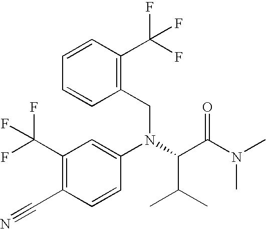 |
| Example 10 | N²-[4-cyano-3-(trifluoromethyl)phenyl]-N¹,N¹-dimethyl-N²-(2-methyl-2-propen-1-yl)-L-valinamide | 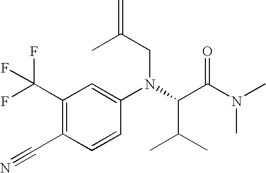 |
| Example 11 | N²-[4-cyano-3-(trifluoromethyl)phenyl]-N¹,N¹-dimethyl-N²-{[2-(trifluoromethyl)phenyl]methyl}-L-isoleucinamide | 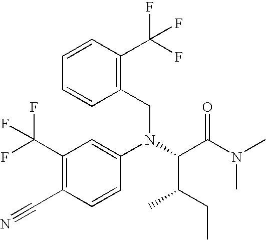 |
| Example 12 | N²-[4-cyano-3-(trifluoromethyl)phenyl]-N¹,N¹-dimethyl-N²-(1-phenylethyl)-L-isoleucinamide | 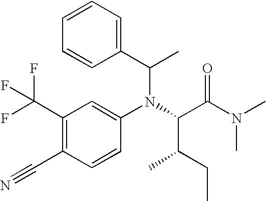 |
| Example 13 | N²-[4-cyano-3-(trifluoromethyl)phenyl]-N¹,N¹-dimethyl-N²-(2-methylpropyl)-L-valinamide | 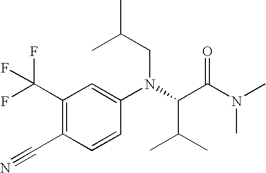 |
| Example 14 | N²-[(2-chlorophenyl)methyl]-N²-[4-cyano-3-(trifluoromethyl)phenyl]-N¹,N¹-dimethyl-D-alaninamide | 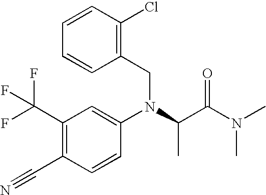 |
| Example 15 | N²-[4-cyano-3-(trifluoromethyl)phenyl]-N²-2-cyclohexen-1-yl-N¹,N¹-dimethyl-L-alaninamide | 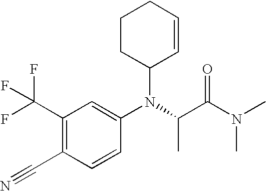 |
| Example 16 | N²-[4-cyano-3-(trifluoromethyl)phenyl]-N²-cyclohexyl-N¹,N¹-dimethyl-L-alaninamide | 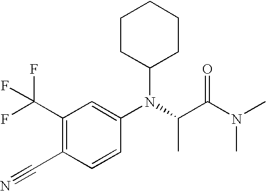 |
| Example 17 | N²-[(2-chlorophenyl)methyl]-N²-[4-cyano-3-(trifluoromethyl)phenyl]-3-cyclohexyl-N¹,N¹-dimethyl-L-alaninamide | 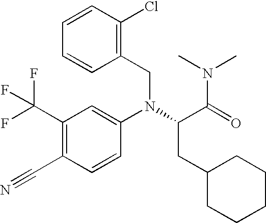 |
| Example 18 | 2-{[(2-chlorophenyl)methyl][4-cyano-3-(trifluoromethyl)phenyl]amino}-N,N-dimethyl-2-phenylacetamide | 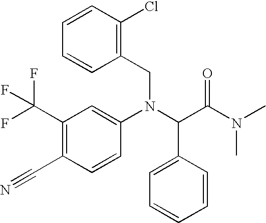 |
| Example 19 | N²-[(2-chlorophenyl)methyl]-N²-[4-cyano-3-(trifluoromethyl)phenyl]-N¹,N¹-dimethyl-D-valinamide | 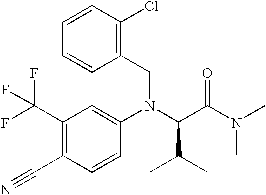 |
| Example 20 | N-[(2-chlorophenyl)methyl]-N-[4-cyano-3-(trifluoromethyl)phenyl]-N,N,O-trimethyl-3-(methyloxy)-L-tyrosinamide | 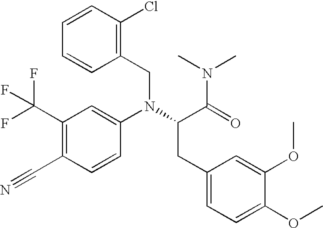 |
| Example 21 | N²-[(2-chlorophenyl)methyl]-N²-[4-cyano-3-(trifluoromethyl)phenyl]-N¹,N¹-dimethyl-3-(2-pyridinyl)alaninamide | 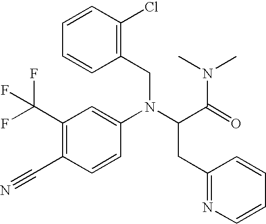 |
| Example 22 | N²-[(2-chlorophenyl)methyl]-N²-[4-cyano-3-(trifluoromethyl)phenyl]-N¹,N¹-dimethyl-L-leucinamide | 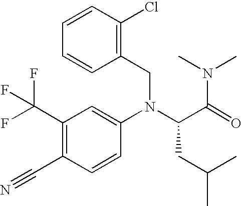 |
| Example 23 | N²-[(2-chlorophenyl)methyl]-N²-[4-cyano-3-(trifluoromethyl)phenyl]-N¹,N¹-dimethyl-D-leucinamide | 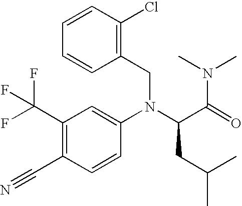 |
| Example 24 | N²-[(2-chlorophenyl)methyl]-N²-[4-cyano-3-(trifluoromethyl)phenyl]-N¹,N¹-dimethyl-D-norleucinamide | 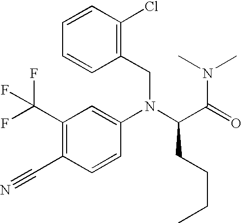 |
| Example 25 | N²-[(2-chlorophenyl)methyl]-N²-[4-cyano-3-(trifluoromethyl)phenyl]-N¹,N¹-dimethyl-L-norleucinamide | 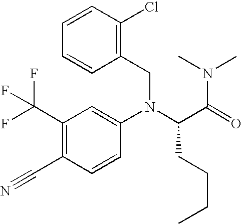 |
| Example 26 | (2S)-2-{[(2-chlorophenyl)methyl][4-cyano-3-(trifluoromethyl)phenyl]amino}-N,N-dimethylbutanamide | 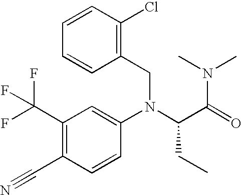 |
| Example 27 | (2R)-2-{[(2-chlorophenyl)methyl][4-cyano-3-(trifluoromethyl)phenyl]amino}-N,N-dimethylbutanamide | 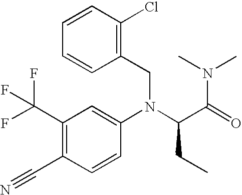 |
| Example 28 | N²-[4-cyano-3-(trifluoromethyl)phenyl]-N²-(2,2-dimethylpropyl)-N¹,N¹-dimethyl-L-valinamide | 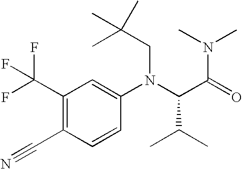 |
| Example 29 | N²-[(2-chlorophenyl)methyl]-N²-(4-cyano-3-fluorophenyl)-N¹,N¹-dimethylalaninamide | 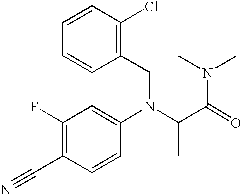 |
| Example 30 | N²-[4-cyano-3-(trifluoromethyl)phenyl]-N¹,N¹-dimethyl-N²-{[2-(methyloxy)phenyl]methyl}alaninamide | 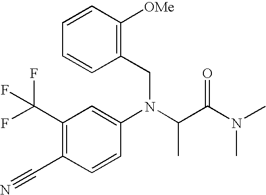 |
| Example 31 | N²-[4-cyano-3-(trifluoromethyl)phenyl]-N¹,N¹-dimethyl-N²-[(2-methylphenyl)methyl]alaninamide | 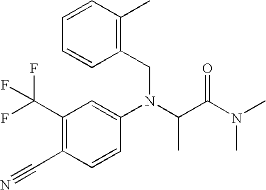 |
| Example 32 | N²-[4-cyano-3-(trifluoromethyl)phenyl]-N²-(2-furanylmethyl)-N¹,N¹-dimethylalaninamide | 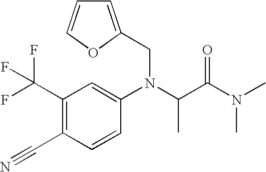 |
| Example 33 | N²-[4-cyano-3-(trifluoromethyl)phenyl]-N¹,N¹,N²-trimethylalaninamide | 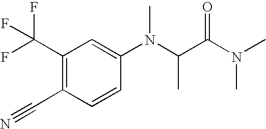 |
| Example 34 | N²-[4-cyano-3-(trifluoromethyl)phenyl]-N²-ethyl-N¹,N¹-dimethylalaninamide | 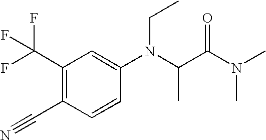 |
| Example 35 | 2-[[4-cyano-3-(trifluoromethyl)phenyl](methyl)amino]-N,N-dimethylbutanamide | 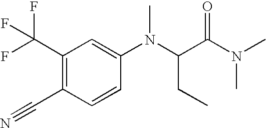 |
| Example 36 | 2-[[4-cyano-3-(trifluoromethyl)phenyl](ethyl)amino]-N,N-dimethylbutanamide | 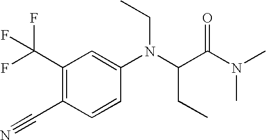 |
| Example 37 | N²-[4-cyano-3-(trifluoromethyl)phenyl]-N²-(2-furanylmethyl)-N¹,N¹-dimethylnorleucinamide | 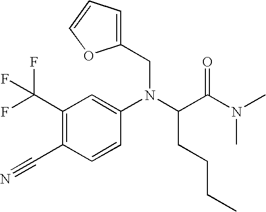 |
| Example 38 | 2-[[4-cyano-3-(trifluoromethyl)phenyl](2-thienylmethyl)amino]-N,N-dimethylbutanamide | 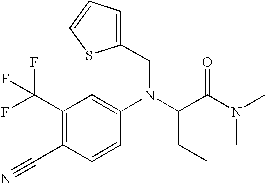 |
| Example 39 | 2-[[4-cyano-3-(trifluoromethyl)phenyl](2-furanylmethyl)amino]-N,N-dimethylbutanamide | 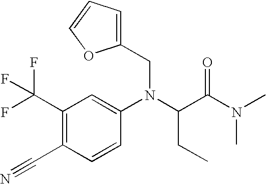 |
| Example 40 | 2-{[(2-chlorophenyl)methyl][4-cyano-3-(trifluoromethyl)phenyl]amino}-N,N-dimethylbutanamide | 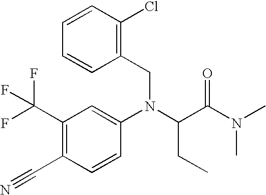 |
| Example 41 | 2-[[4-cyano-3-(trifluoromethyl)phenyl](2-pyridinylmethyl)amino]-N,N-dimethylbutanamide | 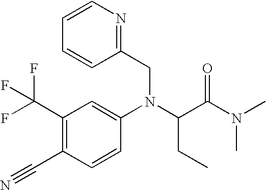 |
| Example 42 | N²-[4-cyano-3-(trifluoromethyl)phenyl]-N²-[(2-fluorophenyl)methyl]-N¹,N¹-dimethylalaninamide | 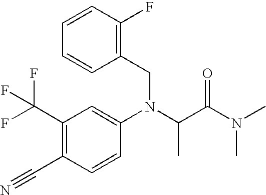 |
| Example 43 | N²-[4-cyano-3-(trifluoromethyl)phenyl]-N¹,N¹-dimethyl-N²-{[3-(trifluoromethyl)phenyl]methyl}alaninamide | 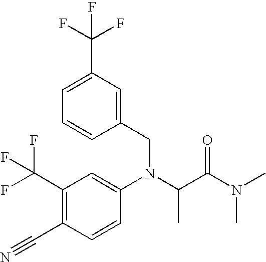 |
| Example 44 | N²-[4-cyano-3-(trifluoromethyl)phenyl]-N¹,N¹-dimethyl-N²-{[4-(trifluoromethyl)phenyl]methyl}alaninamide | 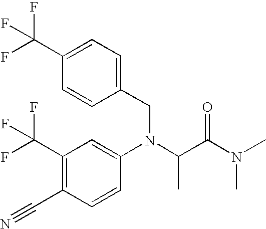 |
| Example 45 | N²-[4-cyano-3-(trifluoromethyl)phenyl]-N¹,N¹-dimethyl-N²-(phenylmethyl)alaninamide | 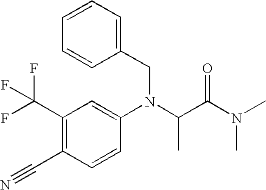 |
| Example 46 | 2-([4-cyano-3-(trifluoromethyl)phenyl]{[2-(methyloxy)phenyl]methyl}amino)-N,N-dimethylbutanamide | 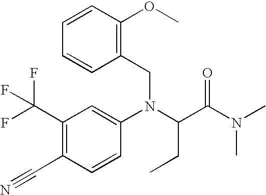 |
| Example 47 | 2-{[4-cyano-3-(trifluoromethyl)phenyl][(2-methylphenyl)methyl]amino}-N,N-dimethylbutanamide | 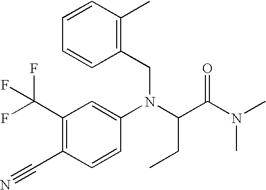 |
| Example 48 | 2-{[4-cyano-3-(trifluoromethyl)phenyl][(2-fluorophenyl)methyl]amino}-N,N-dimethylbutanamide | 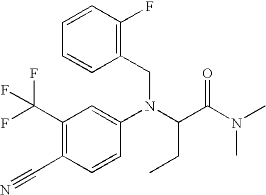 |
| Example 49 | 2-([4-cyano-3-(trifluoromethyl)phenyl]{[3-(trifluoromethyl)phenyl]methyl}amino)-N,N-dimethylbutanamide | 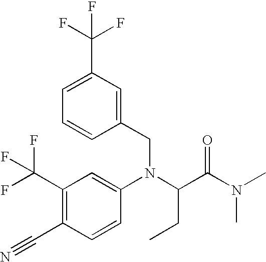 |
| Example 50 | 2-([4-cyano-3-(trifluoromethyl)phenyl]{[4-(trifluoromethyl)phenyl]methyl}amino)-N,N-dimethylbutanamide | 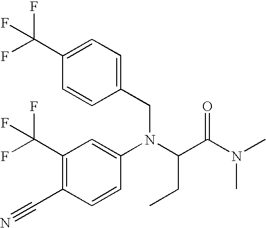 |
| Example 51 | 2-[[4-cyano-3-(trifluoromethyl)phenyl](phenylmethyl)amino]-N,N-dimethylbutanamide | 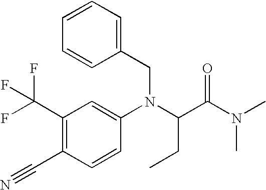 |
| Example 52 | N²-[4-cyano-3-(trifluoromethyl)phenyl]-N¹,N¹-dimethyl-N²-(2-thienylmethyl)alaninamide | 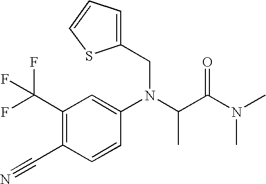 |
| Example 53 | 2-{{[3,4-bis(methyloxy)phenyl]methyl}[4-cyano-3-(trifluoromethyl)phenyl]amino}-N,N-dimethylbutanamide | 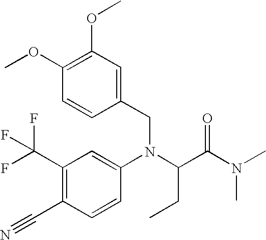 |
| Example 54 | 2-{[4-cyano-3-(trifluoromethyl)phenyl][(3-methyl-2-thienyl)methyl]amino}-N,N-dimethylbutanamide | 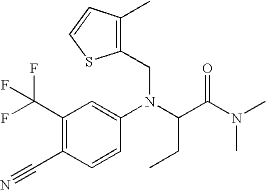 |
| Example 55 | 2-{[4-cyano-3-(trifluoromethyl)phenyl][(5-methyl-2-furanyl)methyl]amino}-N,N-dimethylbutanamide | 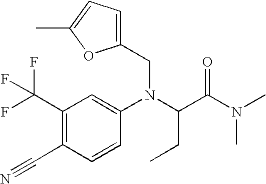 |
| Example 56 | N²-(3-chloro-4-cyanophenyl)-N¹,N¹-dimethyl-N²-(2-phenylethyl)alaninamide | 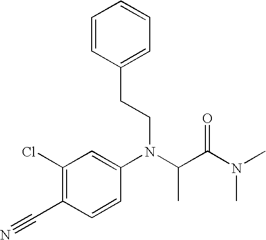 |
| Example 57 | N²-(3-chloro-4-cyanophenyl)-N²-[2-(2-chlorophenyl)ethyl]-N¹,N¹-dimethylalaninamide | 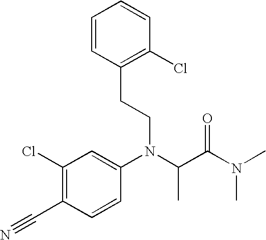 |
| Example 58 | N²-(3-chloro-4-cyanophenyl)-N¹,N¹-dimethyl-N²-(2-thienylmethyl)norleucinamide | 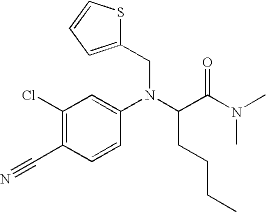 |
| Example 59 | N²-[4-cyano-3-(trifluoromethyl)phenyl]-N¹,N¹-dimethyl-N²-(2,2,2-trifluoroethyl)norvalinamide | 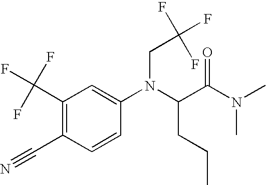 |
| Example 60 | N²-[4-cyano-3-(trifluoromethyl)phenyl]-N¹,N¹-dimethyl-N²-(2,2,2-trifluoroethyl)norleucinamide | 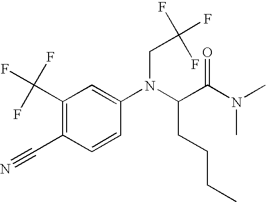 |
| Example 61 | N²-[4-cyano-3-(trifluoromethyl)phenyl]-N¹,N¹-bis(2-methylpropyl)-N²-(2,2,2-trifluoroethyl)alaninamide | 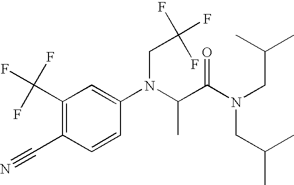 |
| Example 62 | N¹,N¹-dibutyl-N²-[4-cyano-3-(trifluoromethyl)phenyl]-N²-(2,2,2-trifluoroethyl)alaninamide | 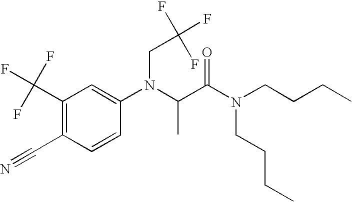 |
| Example 63 | N²-[4-cyano-3-(trifluoromethyl)phenyl]-N¹-(1,1-dimethylethyl)-N¹-methyl-N²-(2,2,2-trifluoroethyl)alaninamide | 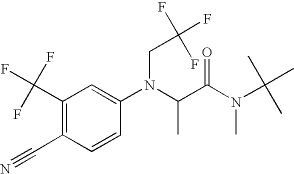 |
| Example 64 | N²-[4-cyano-3-(trifluoromethyl)phenyl]-N¹-methyl-N¹-2-propyn-1-yl-N²-(2,2,2-trifluoroethyl)alaninamide | 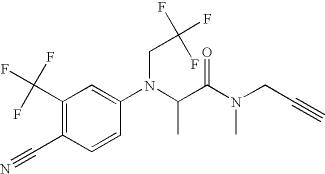 |
| Example 65 | N²-[4-cyano-3-(trifluoromethyl)phenyl]-N¹-ethyl-N¹-propyl-N²-(2,2,2-trifluoroethyl)alaninamide | 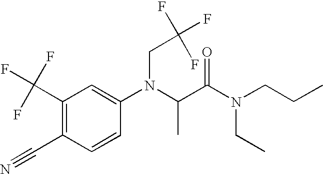 |
| Example 66 | N²-[4-cyano-3-(trifluoromethyl)phenyl]-N¹-ethyl-N¹-(1-methylethyl)-N²-(2,2,2-trifluoroethyl)alaninamide | 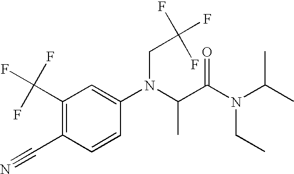 |
| Example 67 | N²-[4-cyano-3-(trifluoromethyl)phenyl]-N¹-ethyl-N¹-methyl-N²-(2,2,2-trifluoroethyl)alaninamide | 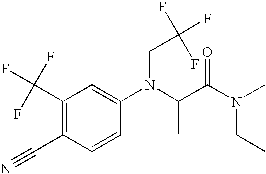 |
| Example 68 | N²-[4-cyano-3-(trifluoromethyl)phenyl]-N¹,N¹-dipropyl-N²-(2,2,2-trifluoroethyl)alaninamide | 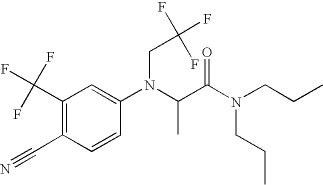 |
| Example 69 | N²-[4-cyano-3-(trifluoromethyl)phenyl]-N¹,N¹-diethyl-N²-(2,2,2-trifluoroethyl)alaninamide | 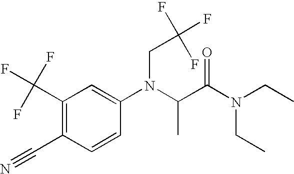 |
| Example 70 | 4-[[1-methyl-2-oxo-2-(1-piperidinyl)ethyl](2,2,2-trifluoroethyl)amino]-2-(trifluoromethyl)benzonitrile | 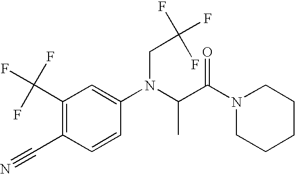 |
| Example 71 | 4-[[2-(1-azetidinyl)-1-methyl-2-oxoethyl](2,2,2-trifluoroethyl)amino]-2-(trifluoromethyl)benzonitrile | 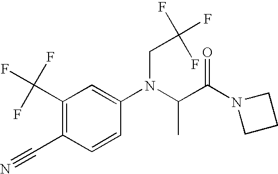 |
| Example 72 | N²-[4-cyano-3-(trifluoromethyl)phenyl]-N¹-methyl-N¹-propyl-N²-(2,2,2-trifluoroethyl)alaninamide | 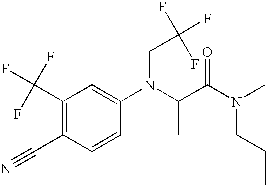 |
| Example 73 | N¹-butyl-N¹-(cyanomethyl)-N²-[4-cyano-3-(trifluoromethyl)phenyl]-N²-(2,2,2-trifluoroethyl)alaninamide | 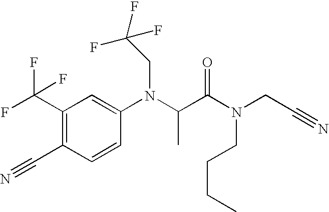 |
| Example 74 | N²-[4-cyano-3-(trifluoromethyl)phenyl]-N¹-[2-(methyloxy)ethyl]-N¹-propyl-N²-(2,2,2-trifluoroethyl)alaninamide | 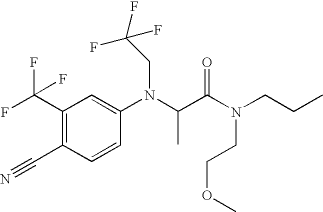 |
| Example 75 | N¹,N¹-bis(cyanomethyl)-N²-[4-cyano-3-(trifluoromethyl)phenyl]-N²-(2,2,2-trifluoroethyl)alaninamide | 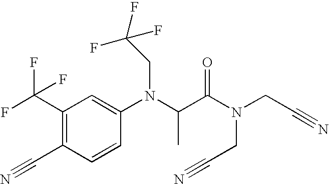 |
| Example 76 | 4-[[1-methyl-2-(4-morpholinyl)-2-oxoethyl](2,2,2-trifluoroethyl)amino]-2-(trifluoromethyl)benzonitrile | 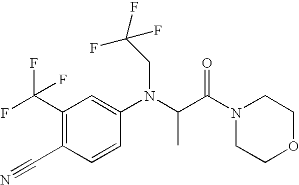 |
| Example 77 | N²-[4-cyano-3-(trifluoromethyl)phenyl]-N¹-(cyclopropylmethyl)-N¹-propyl-N²-(2,2,2-trifluoroethyl)alaninamide | 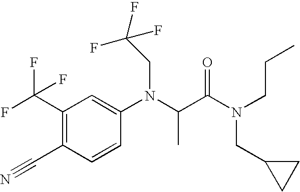 |
| Example 78 | N²-[4-cyano-3-(trifluoromethyl)phenyl]-N¹-cyclohexyl-N¹-methyl-N²-(2,2,2-trifluoroethyl)alaninamide | 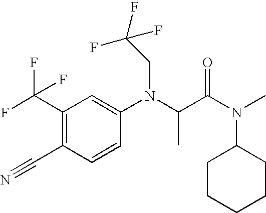 |
| Example 79 | N²-[4-cyano-3-(trifluoromethyl)phenyl]-N¹-methyl-N¹-[2-(methyloxy)ethyl]-N²-(2,2,2-trifluoroethyl)alaninamide | 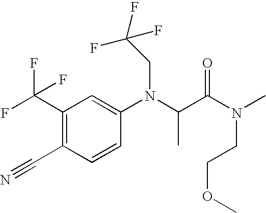 |
| Example 80 | 4-[[1-methyl-2-oxo-2-(4-thiomorpholinyl)ethyl](2,2,2-trifluoroethyl)amino]-2-(trifluoromethyl)benzonitrile | 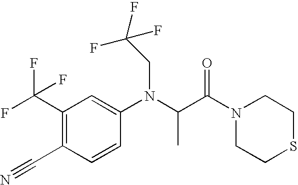 |
| Example 81 | N¹-(2-cyanoethyl)-N²-[4-cyano-3-(trifluoromethyl)phenyl]-N¹-methyl-N²-(2,2,2-trifluoroethyl)alaninamide | 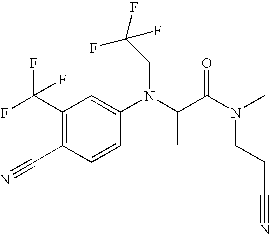 |
| Example 82 | 4-[[1-methyl-2-oxo-2-(1-pyrrolidinyl)ethyl](2,2,2-trifluoroethyl)amino]-2-(trifluoromethyl)benzonitrile | 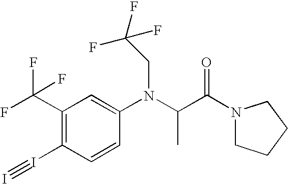 |
| Example 83 | N²-[4-cyano-3-(trifluoromethyl)phenyl]-N¹-methyl-N¹-(1-methylethyl)-N²-(2,2,2-trifluoroethyl)alaninamide | 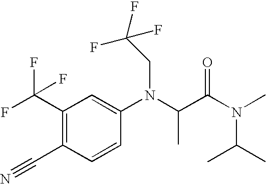 |
| Example 84 | N²-[4-cyano-3-(trifluoromethyl)phenyl]-N¹,N¹-di-2-propen-1-yl-N²-(2,2,2-trifluoroethyl)alaninamide | 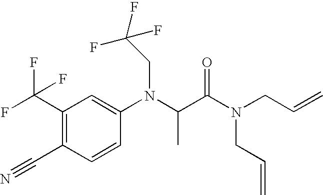 |
| Example 85 | N²-[4-cyano-3-(trifluoromethyl)phenyl]-N¹-methyl-N¹-2-propen-1-yl-N²-(2,2,2-trifluoroethyl)alaninamide | 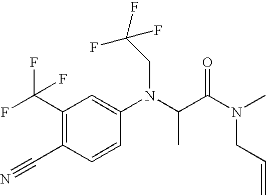 |
| Example 86 | N,N-dibutyl-2-[[4-cyano-3-(trifluoromethyl)phenyl](2,2,2-trifluoroethyl)amino]butanamide | 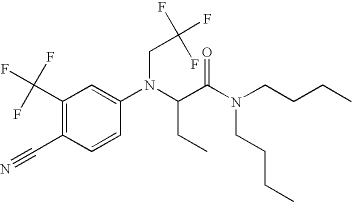 |
| Example 87 | 2-[[4-cyano-3-(trifluoromethyl)phenyl](2,2,2-trifluoroethyl)amino]-N-(1,1-dimethylethyl)-N-methylbutanamide | 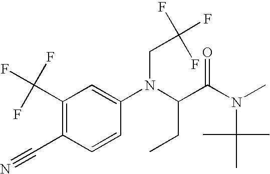 |
| Example 88 | 2-[[4-cyano-3-(trifluoromethyl)phenyl](2,2,2-trifluoroethyl)amino]-N-methyl-N-2-propyn-1-ylbutanamide | 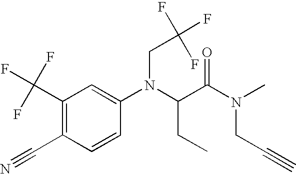 |
| Example 89 | 2-[[4-cyano-3-(trifluoromethyl)phenyl](2,2,2-trifluoroethyl)amino]-N-ethyl-N-propylbutanamide | 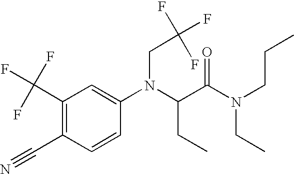 |
| Example 90 | 2-[[4-cyano-3-(trifluoromethyl)phenyl](2,2,2-trifluoroethyl)amino]-N-ethyl-N-(1-methylethyl)butanamide | 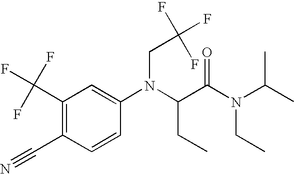 |
| Example 91 | 2-[[4-cyano-3-(trifluoromethyl)phenyl](2,2,2-trifluoroethyl)amino]-N,N-bis(2-methylpropyl)butanamide | 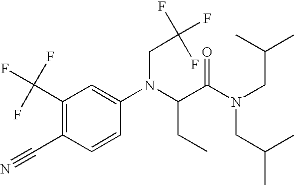 |
| Example 92 | 2-[[4-cyano-3-(trifluoromethyl)phenyl](2,2,2-trifluoroethyl)amino]-N-ethyl-N-methylbutanamide | 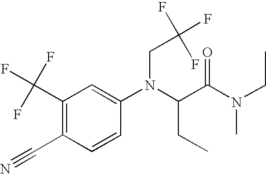 |
| Example 93 | 2-[[4-cyano-3-(trifluoromethyl)phenyl](2,2,2-trifluoroethyl)amino]-N,N-dipropyl-butanamide | 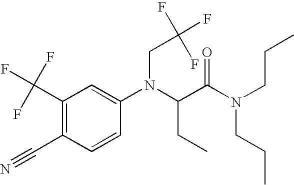 |
| Example 94 | 2-[[4-cyano-3-(trifluoromethyl)phenyl](2,2,2-trifluoroethyl)amino]-N,N-diethyl-butanamide | 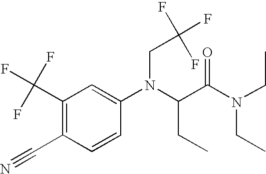 |
| Example 95 | 4-[[1-(1-piperidinylcarbonyl)propyl](2,2,2-trifluoroethyl)amino]-2-(trifluoromethyl)benzonitrile | 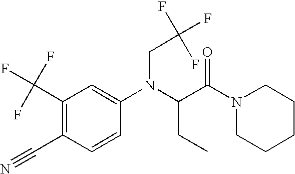 |
| Example 96 | 4-[[1-(1-azetidinylcarbonyl)propyl](2,2,2-trifluoroethyl)amino]-2-(trifluoromethyl)-benzonitrile | 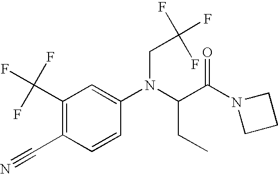 |
| Example 97 | 2-[[4-cyano-3-(trifluoromethyl)phenyl](2,2,2-trifluoroethyl)amino]-N-methyl-N-propylbutanamide | 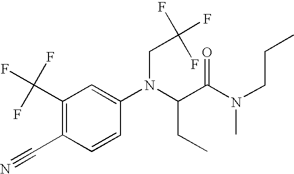 |
| Example 98 | N-butyl-N-(cyanomethyl)-2-[[4-cyano-3-(trifluoromethyl)phenyl](2,2,2-trifluoroethyl)amino]butanamide | 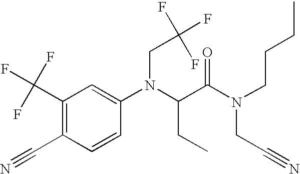 |
| Example 99 | 2-[[4-cyano-3-(trifluoromethyl)phenyl](2,2,2-trifluoroethyl)amino]-N-[2-(methyloxy)ethyl]-N-propylbutanamide | 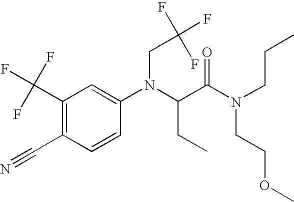 |
| Example 100 | N,N-bis(cyanomethyl)-2-[[4-cyano-3-(trifluoromethyl)phenyl](2,2,2-trifluoroethyl)amino]butanamide | 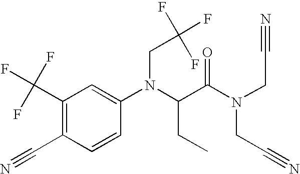 |
| Example 101 | 4-[[1-(4-morpholinylcarbonyl)propyl](2,2,2-trifluoroethyl)amino]-2-(trifluoromethyl)benzonitrile | 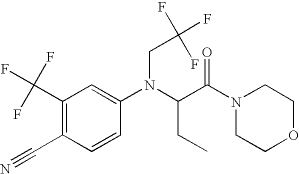 |
| Example 102 | 2-[[4-cyano-3-(trifluoromethyl)phenyl](2,2,2-trifluoroethyl)amino]-N-(cyclopropylmethyl)-N-propylbutanamide | 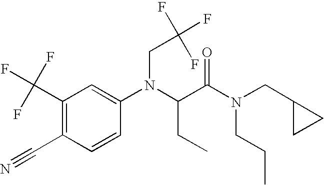 |
| Example 103 | 2-[[4-cyano-3-(trifluoromethyl)phenyl](2,2,2-trifluoroethyl)amino]-N-cyclohexyl-N-methylbutanamide | 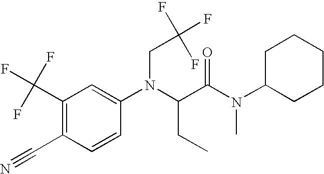 |
| Example 104 | 4-[[1-(4-thiomorpholinylcarbonyl)propyl](2,2,2-trifluoroethyl)amino]-2-(trifluoromethyl)benzonitrile | 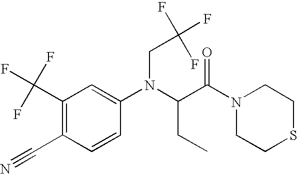 |
| Example 105 | 2-[[4-cyano-3-(trifluoromethyl)phenyl](2,2,2-trifluoroethyl)amino]-N-methyl-N-[2-(methyloxy)ethyl]butanamide | 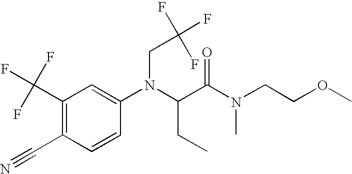 |
| Example 106 | N-(2-cyanoethyl)-2-[[4-cyano-3-(trifluoromethyl)phenyl](2,2,2-trifluoroethyl)-amino]-N-methylbutanamide | 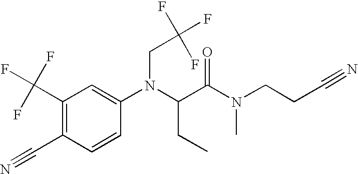 |
| Example 107 | 4-[[1-(1-pyrrolidinylcarbonyl)propyl](2,2,2-trifluoroethyl)amino]-2-(trifluoromethyl)benzonitrile | 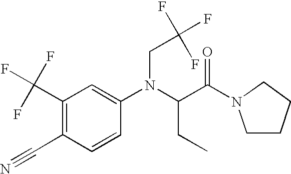 |
| Example 108 | 2-[[4-cyano-3-(trifluoromethyl)phenyl](2,2,2-trifluoroethyl)amino]-N-methyl-N-(1-methylethyl)butanamide | 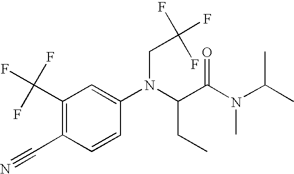 |
| Example 109 | 2-[[4-cyano-3-(trifluoromethyl)phenyl](2,2,2-trifluoroethyl)amino]-N,N-di-2-propen-1-ylbutanamide | 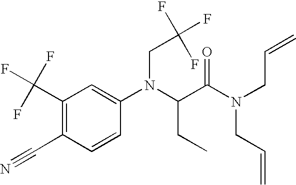 |
| Example 110 | 4-[[1-(1,3-thiazolidin-3-ylcarbonyl)propyl](2,2,2-trifluoroethyl)amino]-2-(trifluoromethyl)benzonitrile | 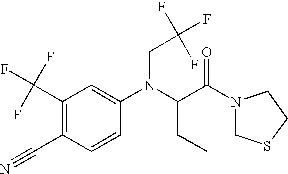 |
| Example 111 | 2-[[4-cyano-3-(trifluoromethyl)phenyl](2,2,2-trifluoroethyl)amino]-N-methyl-N-2-propen-1-ylbutanamide | 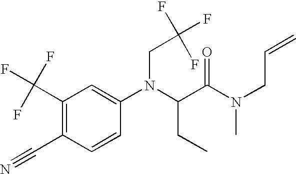 |
| Example 112 | N²-[4-cyano-3-(trifluoromethyl)phenyl]-N¹,N¹-dimethyl-N²-{[2-(trifluoromethyl)-phenyl]methyl}alaninamide | 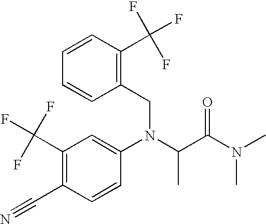 |
| Example 113 | N²-[4-cyano-3-(trifluoromethyl)phenyl]-N²-(2,2-dimethylpropyl)-N¹,N¹-dimethylalaninamide | 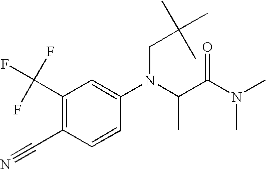 |
| Example 114 | N²-[4-cyano-3-(trifluoromethyl)phenyl]-N²-(3,3-dimethylbutyl)-N¹,N¹-dimethylalaninamide | 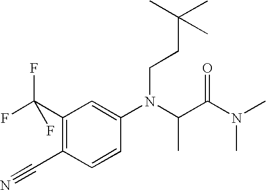 |
| Example 115 | N²-[4-cyano-3-(trifluoromethyl)phenyl]-N¹,N¹-dimethyl-N²-(3,3,3-trifluoropropyl)-alaninamide | 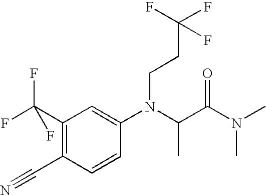 |
| Example 116 | N²-[2,2-bis(methyloxy)ethyl]-N²-[4-cyano-3-(trifluoromethyl)phenyl]-N¹,N¹-dimethylalaninamide | 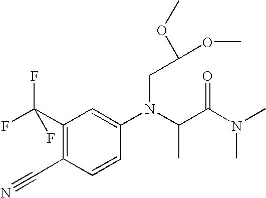 |
| Example 117 | 2-[[4-cyano-3-(trifluoromethyl)phenyl](2,2-dimethylpropyl)amino]-N,N-dimethylbutanamide | 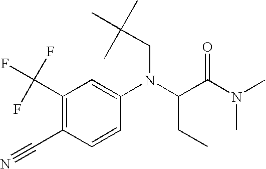 |
| Example 118 | N²-[(2-chlorophenyl)methyl]-N²-(4-cyanophenyl)-N¹,N¹-dimethylalaninamide | 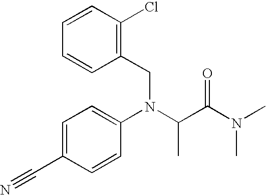 |
| Example 119 | N²-[4-cyano-3-(trifluoromethyl)phenyl]-N²-(2,2-dimethylpropyl)-N¹,N¹-dimethylnorleucinamide | 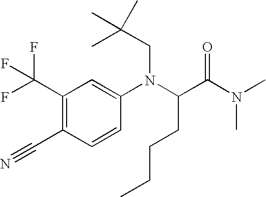 |
| Example 120 | N²-(3-chloro-4-cyanophenyl)-N²-ethyl-N¹,N¹-dimethylalaninamide | 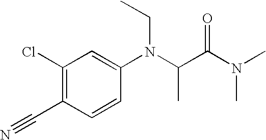 |
| Example 121 | N²-(3-chloro-4-cyanophenyl)-N²-[(2-chlorophenyl)methyl]-N¹,N¹-dimethylalaninamide | 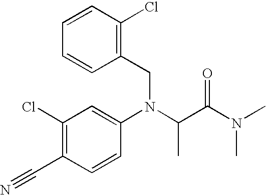 |
| Example 122 | N²-[4-cyano-3-(trifluoromethyl)phenyl]-N²-ethyl-N¹,N¹,2-trimethylalaninamide | 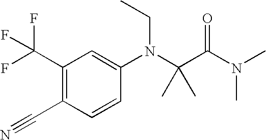 |

Compounds of the present invention can be prepared, for example, as illustrated in Scheme I. In the scheme, Cbz refers to benzyloxycarbonyl; EDC refers to 1-ethyl-3-(3-dimethylaminopropyl)carbodiimide hydrochloride; DMAP refers to dimethylaminopyridine; EtOH refers to ethanol; and DMF refers to dimethylformamide. R^{o}, R¹, R^{1′}, R², R^{2′}, R³, and n are as previously defined.

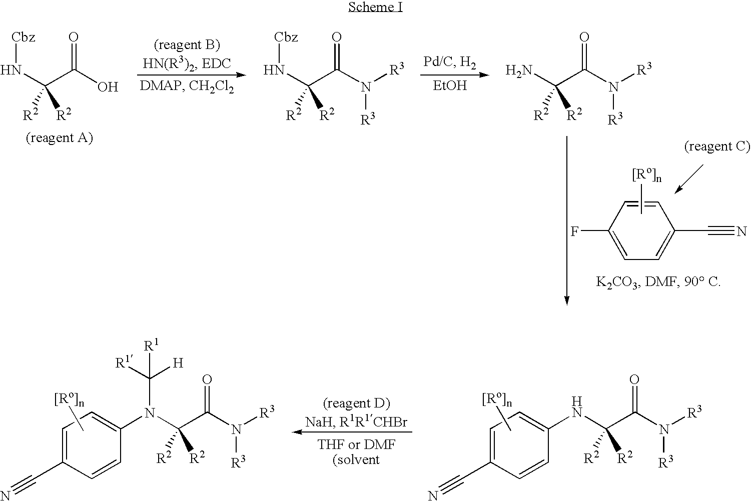

Examples 1-16 were prepared using Scheme I:

### Example 1

### N²-[(2-chlorophenyl)methyl]-N²-[4-cyano-3-(trifluoromethyl)phenyl]-N¹,N¹-dimethyl-L-alaninamide

### A. Phenylmethyl [(1S)-2-(dimethylamino)-1-methyl-2-oxoethyl]carbamate

To an ice cooled solution of N-{[(phenylmethyl)oxy]carbonyl}-L-alanine (5 g, 22.4 mmol) in dichloromethane was added dimethylamine (2.0 M in THF, 19.0 mL, 38.1 mmol), dimethylaminopyridine (0.27 g, 2.24 mmol), and 1-ethyl-3-(3-dimethylaminopropyl)carbodiimide hydrochloride (7.3 g, 38.1 mmol). The reaction was warmed to room temperature and stirred overnight. The reaction was extracted two times with 1 N HCl. The organic layer was dried over Na₂SO₄, concentrated, and the title compound was purified using silica chromatography. ¹H NMR (400 MHz, CDCl₃) δ 7.33 (m, 5H), 5.85 (d, 1H, J=7.2 Hz), 5.11 (s, 2H), 4.69 (quintet, 1H, J=7.2 Hz), 3.08 (s, 3H), 2.98 (s, 3H), 1.34 (d, 3H, J=6.8 Hz). MS m/z 251 (M⁺+1).

### B. N¹,N¹-dimethyl-L-alaninamide

To phenylmethyl [(1S)-2-(dimethylamino)-1-methyl-2-oxoethyl]carbamate (4.93 g, 19.7 mmol) was added 0.74 g of 10% palladium on carbon and 200 mL of ethanol. A hydrogen balloon was attached, and the reaction was stirred overnight. The reaction was filtered through celite, and the filtrate was concentrated to afford the above titled compound. ¹H NMR (400 MHz, MeOH-d₄) δ 4.08 (q, 1H, J=6.8 Hz), 3.10 (s, 3H), 2.98 (s, 3H), 1.32 (d, 3H, J=6.8 Hz). MS m/z 117 (M⁺+1)

### C. N²-[4-cyano-3-(trifluoromethyl)phenyl]-N¹,N¹-dimethyl-L-alaninamide

To a solution of N¹,N¹-dimethyl-L-alaninamide (2.03 g, 17.5 mmol) in 30 mL of DMF was added 4-fluoro-2-(trifluoromethyl)benzonitrile (2.75 g, 14.5 mmol) and potassium carbonate (3.0 g, 21.8 mmol). The reaction was heated at 90° C. for five hours before cooling to room temperature. The reaction was diluted with diethyl ether and extracted with an aqueous LiBr solution and then water. The organic layer was dried over Na₂SO₄, concentrated, and the above titled compound was isolated using silica gel chromatography. ¹H NMR (400 MHz, CDCl₃) δ 7.49 (d, 1H, J=8.4 Hz), 6.84 (s, 1H), 6.64 (dd, 1H, J=1.6, 8.4 Hz), 5.95 (bs, 1H), 4.45 (q, 1H, J=6.4 Hz), 3.14 (s, 3H), 3.02 (s, 3H), 1.40 (d, 3H, J=6.4 Hz). MS m/z 286 (M⁺+1)

### D. N²-[(2-chlorophenyl)methyl]-N²-[4-cyano-3-(trifluoromethyl)phenyl]-N¹,N¹-dimethyl-L-alaninamide

To a suspension of sodium hydride (60% dispersion in mineral oil, 35 mg, 0.876 mmol) in THF or DMF (3.5 mL) at 0° C. was added N²-[4-cyano-3-(trifluoromethyl)phenyl]-N¹,N¹-dimethyl-L-alaninamide (100 mg, 0.35 mmol). The mixture was stirred for 30 min, and then 2-chlorobenzyl bromide (108 mg, 0.525 mmol) was added. The reaction was warmed to room temperature and stirred 4 to 16 hours. (In some circumstances, adding excess reagents or heating at 65-90° C. was necessary to progress a slow reaction.) The reaction was quenched slowly with water and diluted with diethyl ether, and the layers were separated. The organic layer was washed again with water, dried over Na₂SO₄, concentrated, and the above titled compound was isolated using silica gel chromatography. ¹H NMR (400 MHz, CDCl₃) δ 7.55 (d, 1H, J=8.8 Hz), 7.42 (dd, 1H, J=1.6, 7.6 Hz), 7.21 (m, 2H), 7.05 (dd, 1H, J=1.2, 7.6 Hz), 6.88 (d, 1H, J=2.4 Hz), 6.69 (dd, 1H, J=2.8, 9.2 Hz), 4.85 (m, 3H), 3.15 (s, 3H), 2.94 (s, 3H), 1.50 (d, 3H, J=6.8 Hz). MS m/z 410 (M⁺+1).

### Example 2

### N²-[4-cyano-3-(trifluoromethyl)phenyl]-N²-isobutyl-N¹,N¹-dimethyl-L-alaninamide

The above titled compound was prepared according to general sequence as outlined in Scheme I using N-(carbobenzyloxy)-L-alanine (reagent A), dimethylamine (reagent B), 2-(trifluoromethyl)-4-fluorobenzonitrile (reagent C), 1-bromo-2-methylpropane (reagent D) and DMF (solvent). MS m/z 342 (M⁺+1)

### Example 3

### N²-[4-cyano-3-(trifluoromethyl)phenyl]-N¹,N¹-dimethyl-N²-(1-phenylethyl)-L-alaninamide

The above titled compound was prepared according to general sequence as outlined in Scheme I using N-(carbobenzyloxy)-L-alanine (reagent A), dimethylamine (reagent B), 2-(trifluoromethyl)-4-fluorobenzonitrile (reagent C), 1-phenethyl bromide (reagent D) and DMF (solvent). MS m/z 390 (M⁺+1)

### Example 4

### N²-[4-cyano-3-(trifluoromethyl)phenyl]-N¹,N¹-dimethyl-N²-(2-methyl-2-propen-1yl)-L-isoleucinamide

The above titled compound was prepared according to general sequence as outlined in Scheme I using N-(carbobenzyloxy)-L-isoleucine (reagent A), dimethylamine (reagent B), 2-(trifluoromethyl)-4-fluorobenzonitrile (reagent C), 3-bromo-2-methyl-1-propene (reagent D) and DMF (solvent). MS m/z 382 (M⁺+1)

### Example 5

### N²-[4-cyano-3-(trifluoromethyl)phenyl]-N¹,N¹-dimethyl-N²-(1-methylethyl)-L-alaninamide

The above titled compound was prepared according to general sequence as outlined in Scheme I using N-(carbobenzyloxy)-L-alanine (reagent A), dimethylamine (reagent B), 2-(trifluoromethyl)-4-fluorobenzonitrile (reagent C), 2-bromopropane (reagent D) and DMF (solvent). MS m/z 328 (M⁺+1)

### Example 6

### N²-[(2-chlorophenyl)methyl]-N²-[4-cyano-3-(trifluoromethyl)phenyl]-N¹,N¹-dimethyl-L-isoleucinamide

The above titled compound was prepared according to general sequence as outlined in Scheme I using N-(carbobenzyloxy)-L-isoleucine (reagent A), dimethylamine (reagent B), 2-(trifluoromethyl)-4-fluorobenzonitrile (reagent C), 2-chlorobenzyl bromide (reagent D) and DMF (solvent). MS m/z 452 (M⁺+1)

### Example 7

### N²-[4-cyano-3-(trifluoromethyl)phenyl]-N¹,N¹-dimethyl-N²-(2-methylpropyl)-L-isoleucinamide

To N²-[4-cyano-3-(trifluoromethyl)phenyl]-N¹,N¹-dimethyl-N²-(2-methyl-2-propen-1-yl)-L-isoleucinamide (Example 4) (97.5 mg, 0.256 mmol) was added 20 mg of 10% palladium on carbon and 5 mL of ethanol. A hydrogen balloon was attached, and the reaction was stirred overnight. The reaction was filtered through celite, and the filtrate was concentrated. The above titled compound was isolated using silica gel chromatography. ¹H NMR (400 MHz, CDCl₃) δ 7.60 (d, 1H, J=8.4 Hz), 7.10 (s, 1H), 6.97 (d, 1H, J=8.8 Hz), 4.31 (d, 1H, J=10.4 Hz), 3.34 (dd, 1H, J=8.0, 14.8 Hz), 3.15 (dd, 1H, J=6.0, 14.8 Hz), 2.97 (d, 6H, J=3.6 Hz), 2.27 (m, 1H), 1.93 (m, 1H), 1.27 (m, 2H), 0.89 (m, 12H). MS m/z 384 (M⁺+1)

### Example 8

### N²-[(2-chlorophenyl)methyl]-N²-[4-cyano-3-(trifluoromethyl)phenyl]-N¹,N¹-dimethyl-L-valinamide

The above titled compound was prepared according to general sequence as outlined in Scheme I using N-(carbobenzyloxy)-L-valine (reagent A), dimethylamine (reagent B), 2-(trifluoromethyl)-4-fluorobenzonitrile (reagent C), 2-chlorobenzyl bromide (reagent D) and DMF (solvent). MS m/z 438 (M⁺+1)

### Example 9

### N²-[4-cyano-3-(trifluoromethyl)phenyl]-N¹,N¹-dimethyl-N²-{[2-(trifluoromethyl)phenyl]methyl}-L-valinamide

The above titled compound was prepared according to general sequence as outlined in Scheme I using N-(carbobenzyloxy)-L-valine (reagent A), dimethylamine (reagent B), 2-(trifluoromethyl)-4-fluorobenzonitrile (reagent C), 1-(bromomethyl)-2-(trifluoromethyl)benzene (reagent D) and DMF (solvent). MS m/z 472 (M⁺+1)

### Example 10

### N²-[4-cyano-3-(trifluoromethyl)phenyl]-N¹,N¹-dimethyl-N²-(2-methyl-2-propen-1-yl)-L-valinamide

The above titled compound was prepared according to general sequence as outlined in Scheme I using N-(carbobenzyloxy)-L-valine (reagent A), dimethylamine (reagent B), 2-(trifluoromethyl)-4-fluorobenzonitrile (reagent C), 3-bromo-2-methyl-1-propene (reagent D) and DMF (solvent). MS m/z 368 (M⁺+1)

### Example 11

### N²-[4-cyano-3-(trifluoromethyl)phenyl]-N¹,N¹-dimethyl-N²-{[2-(trifluoromethyl)phenyl]methyl}-L-isoleucinamide

The above titled compound was prepared according to general sequence as outlined in Scheme I using N-(carbobenzyloxy)-L-isoleucine (reagent A), dimethylamine (reagent B), 2-(trifluoromethyl)-4-fluorobenzonitrile (reagent C), 1-(bromomethyl)-2-(trifluoromethyl)benzene (reagent D) and DMF (solvent). MS m/z 486 (M⁺+1)

### Example 12

### N²-[4-cyano-3-(trifluoromethyl)phenyl]-N¹,N¹-dimethyl-N²-(1-phenylethyl)-L-isoleucinamide

The above titled compound was prepared according to general sequence as outlined in Scheme I using N-(carbobenzyloxy)-L-isoleucine (reagent A), dimethylamine (reagent B), 2-(trifluoromethyl)-4-fluorobenzonitrile (reagent C), (1-bromoethyl)benzene (reagent D) and DMF (solvent). MS m/z 432 (M⁺+1)

### Example 13

### N²-[4-cyano-3-(trifluoromethyl)phenyl]-N¹,N¹-dimethyl-N²-(2-methylpropyl)-L-valinamide

The above titled compound was prepared using the procedure set forth in Example 7 using N²-[4-cyano-3-(trifluoromethyl)phenyl]-N¹,N¹-dimethyl-N²-(2-methyl-2-propen-1-yl)-L-valinamide (Example 10). MS m/z 370 (M⁺+1)

### Example 14

### N²-[(2-chlorophenyl)methyl]-N²-[4-cyano-3-(trifluoromethyl)phenyl]-N¹,N¹-dimethyl-D-alaninamide

The above titled compound was prepared according to general sequence as outlined in Scheme I using N-(carbobenzyloxy)-D-alanine (reagent A), dimethylamine (reagent B), 2-(trifluoromethyl)-4-fluorobenzonitrile (reagent C), 2-chlorobenzyl bromide (reagent D) and DMF (solvent). MS m/z 410 (M⁺+1)

### Example 15

### N²-[4-cyano-3-(trifluoromethyl)phenyl]-N²-2-cyclohexen-1-yl-N¹,N¹-dimethyl-L-alaninamide

The above titled compound was prepared according to general sequence as outlined in Scheme I using N-(carbobenzyloxy)-L-alanine (reagent A), dimethylamine (reagent B), 2-(trifluoromethyl)-4-fluorobenzonitrile (reagent C), 3-bromocyclohexene (reagent D) and DMF (solvent). MS m/z 366 (M⁺+1)

### Example 16

### N²-[4-cyano-3-(trifluoromethyl)phenyl]-N²-cyclohexyl-N¹,N¹-dimethyl-L-alaninamide

The above titled compound was prepared using the procedure set forth in Example 7 using N²-[4-cyano-3-(trifluoromethyl)phenyl]-N²-2-cyclohexen-1-yl-N¹,N¹-dimethyl-L-alaninamide (Example 15). MS m/z 368 (M⁺+1)

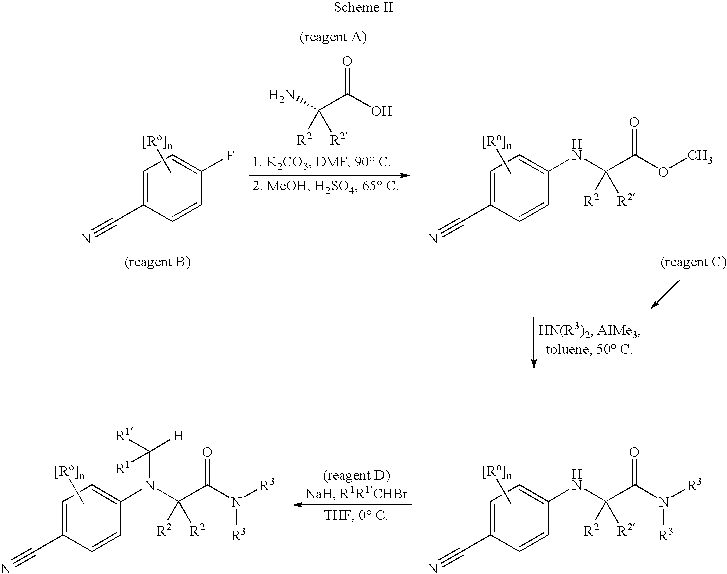

Examples 17-18 were prepared using the procedure set forth in Scheme II.

### Example 17

### N²-[(2-chlorophenyl)methyl]-N²-[4-cyano-3-(trifluoromethyl)phenyl]-3-cyclohexyl-N¹,N¹-dimethyl-L-alaninamide

### A. Methyl N-[4-cyano-3-(trifluoromethyl)phenyl]-3-cyclohexyl-L-alaninate

To a suspension of 3-cyclohexyl-L-alanine (1.36 g, 7.94 mmol) (reagent A) in 80 mL of DMF was added 4-fluoro-2-(trifluoromethyl)benzonitrile (reagent B) (1.0 g, 5.29 mmol) and potassium carbonate (2.19 g, 15.9 mmol). The reaction mixture was heated at 90° C. overnight and cooled to room temperature, diluted with an aqueous LiBr solution, and acidified to pH 3-4 with 6 N HCl. The aqueous layer was extracted three times with ethyl acetate. The combined organic layers were dried over Na₂SO₄ and concentrated. The crude residue was dissolved in MeOH (50 mL), and 1 mL of concentrated sulfuric acid was added. This mixture was heated at 65° C. for five hours. The reaction was cooled to room temperature and concentrated. The residue was slowly quenched with saturated NaHCO₃ and diluted with diethyl ether. The layers were separated, and the organic layer was washed with an aqueous LiBr solution and water. The organic layer was dried over Na₂SO₄, concentrated, and purified by silica chromatography to afford the title compound. ¹H NMR (400 MHz, CDCl₃) δ 7.55 (d, 1H, J=8.8 Hz), 6.89 (d, 1H, J=2.4 Hz), 6.69 (dd, 1H, J=2.4, 8.4 Hz), 5.16 (d, 1H, J=8.4 Hz), 4.17 (q, 1H, J=5.6 Hz), 3.76 (s, 3H), 1.71 (m, 7H), 1.43 (m, 1H), 1.22 (m, 3H), 0.97 (m, 2H). MS m/z 355 (M⁺+1)

### B. N²-[4-cyano-3-(trifluoromethyl)phenyl]-3-cyclohexyl-N¹,N¹-dimethyl-L-alaninamide

To an ice cooled suspension of dimethylamine hydrochloride (reagent C) (2.65 g, 32.3 mmol) in toluene (34 mL), trimethylaluminum (2.0 M in toluene, 16.2 mL, 32.3 mmol) was added slowly (observed gas evolution), and the solids gradually went into solution. The reaction mixture was warmed to room temperature, and a solution of methyl N-[4-cyano-3-(trifluoromethyl)phenyl]-3-cyclohexyl-L-alaninate (1.44 g, 4.06 mmol) in 10 mL of toluene was added slowly. The reaction mixture was heated at 50° C. for 16 hours, then cooled to 0° C. Excess aluminium reagent was quenched by slow addition of 80 mL of 0.5 N HCl. Ethyl acetate was added, and the mixture was stirred for 30 min. The mixture was neutralized with saturated NaHCO₃. The layers were separated, and the aqueous layer was extracted twice with ethyl acetate. The combined organic layers were dried over Na₂SO₄, concentrated, and the title compound was isolated by silica gel chromatography. ¹H NMR (400 MHz, CDCl₃) δ 7.54 (d, 1H, J=8.8 Hz), 6.85 (d, 1H, J=2.4 Hz), 6.66 (dd, 1H, J=2.4, 8.4 Hz), 5.45 (bs, 1H), 4.46 (t, 1H, J=6.8 Hz), 3.15 (s, 3H), 3.02 (s, 3H), 1.85 (m, 1H), 1.67 (m, 6H), 1.43 (m, 1H), 1.23 (m, 3H), 1.00 (m, 2H). MS m/z 368 (M⁺+1)

### C. N²-[(2-chlorophenyl)methyl]-N²-[4-cyano-3-(trifluoromethyl)phenyl]-3-cyclohexyl-N¹,N¹-dimethyl-L-alaninamide

The title compound was prepared according to the procedure set forth in Example I, Step D from N²-[4-cyano-3-(trifluoromethyl)phenyl]-3-cyclohexyl-N¹,N¹-dimethyl-L-alaninamide (Example 17, Step B, above) and 2-chlorobenzyl bromide (reagent D). MS m/z 492 (M⁺+1)

### Example 18

### 2-{[(2-chlorophenyl)methyl][4-cyano-3-(trifluoromethyl)phenyl]amino}-N,N-dimethyl-2-phenylacetamide

The above titled compound was prepared according to general sequence as outlined in Scheme II using DL-phenylglycine (reagent A), 2-(trifluoromethyl)-4-fluorobenzonitrile (reagent B), dimethylamine (reagent C), and 2-chlorobenzyl bromide (reagent D). MS m/z 472 (M⁺+1)

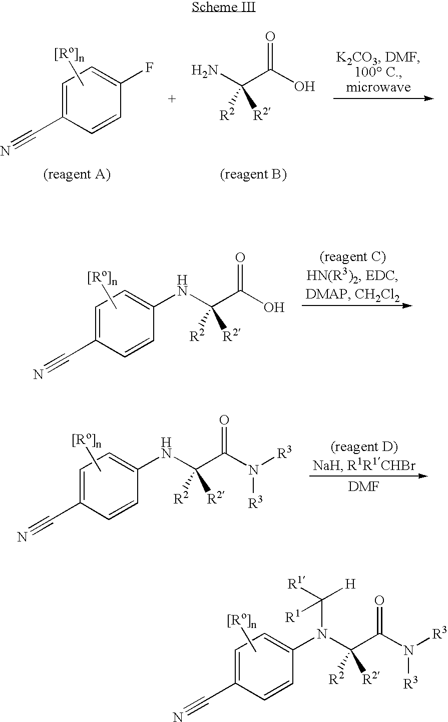

Examples 19-29 were prepared using the sequence outlined in Scheme III.

### Example 19

### N²-[(2-chlorophenyl)methyl]-N²-[4-cyano-3-(trifluoromethyl)phenyl]-N¹,N¹-dimethyl-D-valinamide

### A. N-[4-cyano-3-(trifluoromethyl)phenyl]-D-valine

A mixture of 4-fluoro-2-(trifluoromethyl)benzonitrile (reagent A) (500 mg, 2.64 mmol, 1 eq), D-valine (reagent B) (341 mg, 2.91 mmol, 1.1 eq), and K₂CO₃ (547 mg, 3.96 mmol, 1.5 eq) in DMF (6 mL) was sealed in a 10-20 mL microwave process vial and heated in a microwave reactor for 20 min at 100° C. The reaction mixture was partitioned between ethyl acetate and water and acidified to pH 3 with 1N HCl. The layers were separated, and the organic layer was washed with saturated aqueous sodium carbonate (2-50 mL portions) and dried over sodium sulfate. The residue was purified by ISCO silica gel chromatography (10 g silica cartridge, 100% CH₂Cl₂ grading to 10% CH₃OH/CH₂Cl₂ over 10 min followed by 10% CH₂Cl₂ for 10 min) to provide the title compound (320 mg, 42%) as a pale yellow oil. ¹H NMR (400 MHz, CD₃OD) δ 7.63 (d, 1H, J=8.8 Hz), 7.13 (d, 1H, J=2.0 Hz), 6.88 (dd, 1H, J=8.4, 2.0 Hz), 3.91 (d, 1H, J=6.4 Hz), 2.24 (m, 1H), 1.09 (t, 6H, J=6.8 Hz); LC/MS 287.2 (MH)⁺.

### B. N²-[4-cyano-3-(trifluoromethyl)phenyl]-N¹,N¹-dimethyl-D-valinamide

EDC (249 mg, 1.30 mmol, 1.2 eq) was added in one portion to a solution of N-[4-cyano-3-(trifluoromethyl)phenyl]-D-valine (310 mg, 1.08 mmol, 1 eq), dimethylamine (reagent C) (812 microliters of a 2.0 M solution in THF, 1.62 mmol, 1.5 eq) and DMAP (5 mg) in dichloromethane (5 mL). The resultant pale yellow solution was stirred at room temperature for 36 hours, washed with water (1-25 mL portion), dried over sodium sulfate and concentrated. The residue was purified by ISCO silica gel chromatography (10 g silica cartridge, 100% CH₂Cl₂ grading to 10% CH₃OH/CH₂Cl₂ over 20 min followed by 10% CH₂Cl₂ for 10 min) to provide the title compound (122 mg, 36%) as a white foam. ¹H NMR (400 MHz, CDCl₃) δ 7.55 (d, 1H, J=8.8 Hz), 6.91 (d, 1H, J=2.4 Hz), 6.74 (dd, 1H, J=8.4, 2.4 Hz), 5.32 (br s, 1H), 4.45 (br s, 1H), 3.16 (s, 3H), 3.02 (s, 3H), 2.13 (m, 1H), 1.06 (d, 3H, J=6.8 Hz), 1.00 (d, 3H, J=6.8 Hz); LC/MS 314.2 (MH)⁺.

### C. N²-[(2-chlorophenyl)methyl]-N²-[4-cyano-3-(trifluoromethyl)phenyl]-N¹N¹-dimethyl-D-valinamide

Sodium hydride (44 mg of a 60% dispersion in mineral oil, 1.1 mmol, 3 eq) was added to a cold (0° C.) solution of N²-[4-cyano-3-(trifluoromethyl)phenyl]-N¹,N¹-dimethyl-D-valinamide in DMF (2 mL). The reaction mixture was allowed to warm to room temperature and then stirred at room temperature for 45 min. The bright yellow reaction mixture was then cooled to 0° C., and 2-chlorobenzyl bromide (reagent D) (154 microliters, 226 mg, 1.1 mmol, 3 eq) was added via syringe. The reaction mixture was stirred for 2 h at 0° C., and then excess hydride was quenched by the addition of water (1 mL). The reaction mixture was partitioned between ethyl acetate and water, and the layers were separated. The organic layer was washed with saturated aqueous sodium chloride (3-25 mL portions), and the combined aqueous washed were back-extracted with ethyl acetate (1-25 mL portion). The combined organics were dried over sodium sulfate and were concentrated. The residue was purified by ISCO silica gel chromatography (10 g silica cartridge, 100% CH₂Cl₂ for 5 min, then grading to 10% CH₃OH/CH₂Cl₂ over 15 min, followed by 10% CH₂Cl₂ for 5 min) to provide the title compound (105 mg, 65%) as a white foam. ¹H NMR (400 MHz, CDCl₃) δ 7.57 (d, 1H, J=8.8 Hz), 7.43 (d, 1H, J=1.2 Hz), 7.19 (t, 1H, J=6.4 Hz), 7.11 (m, 2H), 6.81 (dd, 1H, J=6.4, 2.4 Hz), 6.74 (d, 1H, J=7.6 Hz), 5.07 (d, 1H, 18 Hz), 4.58 (t, 1H, 10 Hz), 3.11 (s, 3H), 2.76 (s, 3H), 2.72 (m, 1H), 1.02 (d, 3H, J=6.4 Hz), 0.94 (d, 3H, J=7.2 Hz);

LC/MS 438.4 (MH)⁺.

### Example 20

### N-[(2-chlorophenyl)methyl]-N-[4-cyano-3-(trifluoromethyl)phenyl]-N,N,O-trimethyl-3-(methyloxy)-L-tyrosinamide

The above titled compound was prepared according to general sequence outlined in Scheme III using 4-fluoro-2-(trifluoromethyl)benzonitrile (reagent A), L-3,4-dimethoxyphenylalanine (reagent B), dimethylamine (reagent C), and 2-chlorobenzyl bromide (reagent D). MS m/z 546.2 (M⁺+1).

### Example 21

### N²-[(2-chlorophenyl)methyl]-N²-[4-cyano-3-(trifluoromethyl)phenyl]-N¹,N¹-dimethyl-3-(2-pyridinyl)alaninamide

The above titled compound was prepared according to general sequence outlined in Scheme III using 4-fluoro-2-(trifluoromethyl)benzonitrile (reagent A), 3-(2-pyridinyl)alanine (reagent B), dimethylamine (reagent C), and 2-chlorobenzyl bromide (reagent D). MS m/z 486.6 (M⁺).

### Example 22

### N²-[(2-chlorophenyl)methyl]-N²-[4-cyano-3-(trifluoromethyl)phenyl]-N¹,N¹-dimethyl-L-leucinamide

The above titled compound was prepared according to general sequence outlined in Scheme III using 4-fluoro-2-(trifluoromethyl)benzonitrile (reagent A), L-leucine (reagent B), dimethylamine (reagent C), and 2-chlorobenzyl bromide (reagent D). MS m/z 541.4 (M⁺).

### Example 23

### N²-[(2-chlorophenyl)methyl]-N²-[4-cyano-3-(trifluoromethyl)phenyl]-N¹,N¹-dimethyl-D-leucinamide

The above titled compound was prepared according to general sequence outlined in Scheme III using 4-fluoro-2-(trifluoromethyl)benzonitrile (reagent A), D-leucine (reagent B), dimethylamine (reagent C), and 2-chlorobenzyl bromide (reagent D). MS m/z 541.6 (M⁺).

### Example 24

### N²-[(2-chlorophenyl)methyl]-N²-[4-cyano-3-(trifluoromethyl)phenyl]-N¹,N¹-dimethyl-D-norleucinamide

The above titled compound was prepared according to general sequence outlined in Scheme III using 4-fluoro-2-(trifluoromethyl)benzonitrile (reagent A), D-norleucine (reagent B), dimethylamine (reagent C), and 2-chlorobenzyl bromide (reagent D). MS m/z 541.6 (M⁺).

### Example 25

### N²-[(2-chlorophenyl)methyl]-N²-[4-cyano-3-(trifluoromethyl)phenyl]-N¹,N¹-dimethyl-L-norleucinamide

The above titled compound was prepared according to general sequence outlined in Scheme III using 4-fluoro-2-(trifluoromethyl)benzonitrile (reagent A), L-norleucine (reagent B), dimethylamine (reagent C), and 2-chlorobenzyl bromide (reagent D). MS m/z 541.6 (M⁺).

### Example 26

### (2S)-2-{[(2-chlorophenyl)methyl][4-cyano-3-(trifluoromethyl)phenyl]amino}-N,N-dimethylbutanamide

The above titled compound was prepared according to general sequence outlined in Scheme III using 4-fluoro-2-(trifluoromethyl)benzonitrile (reagent A), (S)-2-aminobutanoic acid (reagent B), dimethylamine (reagent C), and 2-chlorobenzyl bromide (reagent D). MS m/z 424.2 (M⁺+1).

### Example 27

### (2R)-2-{[(2-chlorophenyl)methyl][4-cyano-3-(trifluoromethyl)phenyl]amino}-N,N-dimethylbutanamide

The above titled compound was prepared according to general sequence outlined in Scheme III using 4-fluoro-2-(trifluoromethyl)benzonitrile (reagent A), (R)-2-aminobutanoic acid (reagent B), dimethylamine (reagent C), and 2-chlorobenzyl bromide (reagent D). MS m/z 424.2 (M⁺+1).

### Example 28

### N²-[4-cyano-3-(trifluoromethyl)phenyl]-N²-(2,2-dimethylpropyl)-N¹,N¹-dimethyl-L-valinamide

The above titled compound was prepared according to general sequence outlined in Scheme III using 4-fluoro-2-(trifluoromethyl)benzonitrile (reagent A), L-valine (reagent B), dimethylamine (reagent C), and 1-bromo-2,2-dimethylpropane. MS m/z 384.4 (M⁺+1).

### Example 29

### N²-[(2-chlorophenyl)methyl]-N²-(4-cyano-3-fluorophenyl)-N¹,N¹-dimethylalaninamide

The above titled compound was prepared according to general sequence outlined in Scheme III using 4-fluoro-2-(trifluoromethyl)benzonitrile (reagent A), alanine (reagent B), dimethylamine (reagent C), and 1-(bromomethyl)-2-chlorobenzene (reagent D). MS m/z 360.0 (M⁺+1).

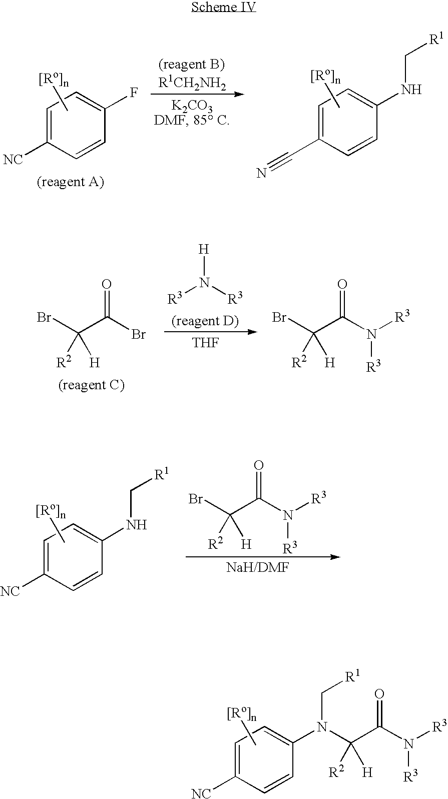

Examples 30-58 were prepared using the sequence outlined in Scheme IV.

### Example 30

### N²-[4-cyano-3-(trifluoromethyl)phenyl]-N¹,N¹-dimethyl-N²-{[2-(methyloxy)phenyl]methyl}alaninamide

### A. 4-({[2-(methyloxy)phenyl]methyl}amino)-2-(trifluoromethyl)benzonitrile

To a solution of 4-flouro-2-(trifluouromethyl)benzonitrile (reagent A) (500 mg, 2.6 mmol) in anhydrous DMF (0.5 M) was added 2-methoxybenzylamine (reagent B) (0.35 mL, 2.6 mmol) and oven-dried potassium carbonate (365 mg, 2.6 mmol). The reaction mixture stirred at 85° C. for 18 hours. After cooling to room temperature, the reaction mixture was diluted with water. The organics were extracted into ethyl ether and dried over sodium sulfate. The filtrate was concentrated, and remaining DMF was removed under reduced pressure to yield a beige solid (415 mg, 51% crude yield): MS (ESI) 306.3 (MH)⁺.

### B. 2-bromo-N,N-dimethylpropanamide

To a solution of dimethylamine (reagent D) (2.0M in THF, 30 mL) was added 2-bromopropionyl bromide (reagent C) (8.7 g, 0.04 mmol) at 0° C. The resulting mixture was kept stirring at 0° C. for additional 10 min. After the reaction was filtered the filtrate was washed with very dilute HCl and dried over sodium sulfate. Solvent was removed to give 5.12 g (70% yield) of the title compound as a colorless oil. MS m/e 180.0 (M+H)⁺.

### C. N²-[4-cyano-3-(trifluoromethyl)phenyl]-N¹,N¹-dimethyl-N²-{[2-(methyloxy)phenyl]methyl}alaninamide

To an ice cold suspension of sodium hydride (60% in mineral oil, 25 mg, 0.64 mmol) in anhydrous DMF was added 4-({[2-(methyloxy)phenyl]methyl}amino)-2-(trifluoromethyl)benzonitrile (Step A) (50.0 mg, 0.16 mmol). After stirring at 0° C. for 30 to 45 min, a solution of 2-bromo-N,N-dimethylpropanamide (Step B) (88.0 mg, 0.49 mmol) in DMF (0.5 mL) was added dropwise to the cold amine solution. After stirring at room temperature for 18 hours, the crude reaction mixture was quenched with H₂O then slowly poured into water. The aqueous phase was extracted three times with diethyl ether, and the organic extracts were combined and concentrated. The oily residue was purified by HPLC (Xterra Prep RP, 100×30 mm, 40 mL/min, A: acetonitrile B: water, A: 10-90% during 10 min, UV detection at 214 nM) to yield 6.5 mg (10% yield) of the title compound as a yellow solid: MS (ESI) 406.0 (MH)⁺; ¹H NMR (400 MHz, CDCl₃) δ 7.56(d, J=8.8 Hz, 1H), 7.27 (d, J=8.8 Hz, 1H), 6.97-6.87 (m, 4H), 6.76 (dd, J=2.8 and 8.8 Hz. 1H), 4.87 (q, 1H), 4.71 (s, 2H), 3.89 (s, 3H), 3.08 (s, 3H), 2.92 (s, 3H), 1.50 (d, J=6.8Hz, 3H)

### Example 31

### N²-[4-cyano-3-(trifluoromethyl)phenyl]-N¹,N¹-dimethyl-N²-[(2-methylphenyl)methyl]alaninamide

The above titled compound was prepared according to general sequence outlined in Scheme IV using 4-fluoro-2-(trifluoromethyl)benzonitrile (reagent A), 2-methylbenzylamine (reagent B), 2-bromopropionyl bromide (reagent C), and dimethylamine (reagent D). MS (ESI) 390.2 (MH)⁺

### Example 32

### N²-[4-cyano-3-(trifluoromethyl)phenyl]-N²-(2-furanylmethyl)-N¹,N¹-dimethylalaninamide

The above titled compound was prepared according to general sequence outlined in Scheme IV using 4-fluoro-2-(trifluoromethyl)benzonitrile (reagent A), (2-furanylmethyl)amine (reagent B), 2-bromopropionyl bromide (reagent C), and dimethylamine (reagent D). MS (ESI) 366.0 (MH)⁺

### Example 33

### N²-[4-cyano-3-(trifluoromethyl)phenyl]-N¹,N¹,N²-trimethylalaninamide

The above titled compound was prepared according to general sequence outlined in Scheme IV using 4-fluoro-2-(trifluoromethyl)benzonitrile (reagent A), methylamine (reagent B), 2-bromopropionyl bromide (reagent C), and dimethylamine (reagent D). MS m/e 300.2 (M+H)⁺.

### Example 34

### N²-[4-cyano-3-(trifluoromethyl)phenyl]-N²-ethyl-N¹,N¹-dimethylalaninamide

The above titled compound was prepared according to general sequence outlined in Scheme IV using 4-fluoro-2-(trifluoromethyl)benzonitrile (reagent A), ethylamine (reagent B), 2-bromopropionyl bromide (reagent C), and dimethylamine (reagent D). MS m/e 300.2 (M+H)⁺.

### Example 35

### 2-[[4-cyano-3-(trifluoromethyl)phenyl](methyl)amino]-N,N-dimethylbutanamide

The above titled compound was prepared according to general sequence outlined in Scheme IV using 4-fluoro-2-(trifluoromethyl)benzonitrile (reagent A), methylamine (reagent B), 2-broniobutyryl bromide (reagent C), and dimethylamine (reagent D). MS m/e 314.2 (M+H)⁺.

### Example 36

### 2-[[4-cyano-3-(trifluoromethyl)phenyl](ethyl)amino]-N,N-dimethylbutanamide

The above titled compound was prepared according to general sequence outlined in Scheme IV using 4-fluoro-2-(trifluoromethyl)benzonitrile (reagent A), ethylamine (reagent B), 2-bromobutyryl bromide (reagent C), and dimethylamine (reagent D). MS m/e 328.2 (M+H)⁺.

### Example 37

### N²-[4-cyano-3-(trifluoromethyl)phenyl]-N²-(2-furanylmethyl)-N¹,N¹-dimethylnorleucinamide

The above titled compound was prepared according to general sequence outlined in Scheme IV using 4-fluoro-2-(trifluoromethyl)benzonitrile (reagent A), (2-furanylmethyl)amine (reagent B), 2-bromohexanoyl bromide (reagent C), and dimethylamine (reagent D). MS m/e 408.2 (M+H)⁺.

### Example 38

### 2-[[4-cyano-3-(trifluoromethyl)phenyl](2-thienylmethyl)amino]-N,N-dimethylbutanamide

The above titled compound was prepared according to general sequence outlined in Scheme IV using 4-fluoro-2-(trifluoromethyl)benzonitrile (reagent A), (2-thienylmethyl)amine (reagent B), 2-bromobutyryl bromide (reagent C), and dimethylamine (reagent D). MS m/e 396.2 (M+H)⁺.

### Example 39

### 2-[[4-cyano-3-(trifluoromethyl)phenyl](2-furanylmethyl)amino]-N,N-dimethylbutanamide

The above titled compound was prepared according to general sequence outlined in Scheme IV using 4-fluoro-2-(trifluoromethyl)benzonitrile (reagent A), (2-furanylmethyl)amine (reagent B), 2-bromobutyryl bromide (reagent C), and dimethylamine (reagent D). MS m/e 380.4 (M+H)⁺.

### Example 40

### 2-{[(2-chlorophenyl)methyl][4-cyano-3-(trifluoromethyl)phenyl]amino}-N,N-dimethylbutanamide

The above titled compound was prepared according to general sequence outlined in Scheme IV using 4-fluoro-2-(trifluoromethyl)benzonitrile (reagent A), [(2-chlorophenyl)methyl]amine (reagent B), 2-bromobutyryl bromide (reagent C), and dimethylamine (reagent D). MS m/e 424.2 (M+H)⁺.

### Example 41

### 2-[[4-cyano-3-(trifluoromethyl)phenyl](2-pyridinylmethyl)amino]-N,N-dimethylbutanamide

The above titled compound was prepared according to general sequence outlined in Scheme IV using 4-fluoro-2-(trifluoromethyl)benzonitrile (reagent A), (2-pyridinylmethyl)amine (reagent B), 2-bromobutyryl bromide (reagent C), and dimethylamine (reagent D). MS m/e 391.2 (M+H)⁺.

### Example 42

### N²-[4-cyano-3-(trifluoromethyl)phenyl]-N²-[(2-fluorophenyl)methyl]-N¹,N¹-dimethylalaninamide

The above titled compound was prepared according to general sequence outlined in Scheme IV using 4-fluoro-2-(trifluoromethyl)benzonitrile (reagent A), [(2-fluorophenyl)methyl]amine (reagent B), 2-bromopropionyl bromide (reagent C), and dimethylamine (reagent D). MS m/e 394.2 (M+M)⁺

### Example 43

### N²-[4-cyano-3-(trifluoromethyl)phenyl]-N¹,N¹-dimethyl-N²-{[3-(trifluoromethyl)phenyl]methyl}alaninamide

The above titled compound was prepared according to general sequence outlined in Scheme IV using 4-fluoro-2-(trifluoromethyl)benzonitrile (reagent A), [(2-trifluoromethylphenyl)methyl]amine (reagent B), 2-bromopropionyl bromide (reagent C), and dimethylamine (reagent D). MS m/e 444.2 (M+H)⁺.

### Example 44

### N²-[4-cyano-3-(trifluoromethyl)phenyl]-N¹,N¹-dimethyl-N²-{[4-(trifluoromethyl)phenyl]methyl}alaninamide

The above titled compound was prepared according to general sequence outlined in Scheme IV using 4-fluoro-2-(trifluoromethyl)benzonitrile (reagent A), [(4-trifluoromethylphenyl)methyl]amine (reagent B), 2-bromopropionyl bromide (reagent C), and dimethylamine (reagent D). MS m/e 444.2 (M+H)⁺

### Example 45

### N²-[4-cyano-3-(trifluoromethyl)phenyl]-N¹,N¹-dimethyl-N²-(phenylmethyl)alaninamide

The above titled compound was prepared according to general sequence outlined in Scheme IV using 4-fluoro-2-(trifluoromethyl)benzonitrile (reagent A), benzylamine (reagent B), 2-bromopropionyl bromide (reagent C), and dimethylamine (reagent D). MS m/e 376.2 (M+H)⁺.

### Example 46

### 2-([4-cyano-3-(trifluoromethyl)phenyl]{[2-(methyloxy)phenyl]methyl}amino)-N,N-dimethylbutanamide

The above titled compound was prepared according to general sequence outlined in Scheme IV using 4-fluoro-2-(trifluoromethyl)benzonitrile (reagent A), [(2-methyloxyphenyl)methyl]amine (reagent B), 2-bromobutanoyl bromide (reagent C), and dimethylamine (reagent D). MS m/e 420.2 (M+H)⁺.

### Example 47

### 2-{[4-cyano-3-(trifluoromethyl)phenyl][(2-methylphenyl)methyl]amino}-N,N-dimethylbutanamide

The above titled compound was prepared according to general sequence outlined in Scheme IV using 4-fluoro-2-(trifluoromethyl)benzonitrile (reagent A), [(2-methylphenyl)methyl]amine (reagent B), 2-bromobutanoyl bromide (reagent C), and dimethylamine (reagent D). MS m/e 404.4 (M+H)⁺.

### Example 48

### 2-{[4-cyano-3-(trifluoromethyl)phenyl][(2-fluorophenyl)methyl]amino}-N,N-dimethylbutanamide

The above titled compound was prepared according to general sequence outlined in Scheme IV using 4-fluoro-2-(trifluoromethyl)benzonitrile (reagent A), [(2-fluorophenyl)methyl]amine (reagent B), 2-bromobutanoyl bromide (reagent C), and dimethylamine (reagent D). MS m/e 408.4 (M+H)⁺.

### Example 49

### 2-([4-cyano-3-(trifluoromethyl)phenyl]{[3-(trifluoromethyl)phenyl]methyl}amino)-N,N-dimethylbutanamide

The above titled compound was prepared according to general sequence outlined in Scheme IV using 4-fluoro-2-(trifluoromethyl)benzonitrile (reagent A), [(3-trifluoromethylphenyl)methyl]amine (reagent B), 2-bromobutanoyl bromide (reagent C), and dimethylamine (reagent D). MS m/e 458.2 (M+H)⁺.

### Example 50

### 2-([4-cyano-3-(trifluoromethyl)phenyl]{[4-(trifluoromethyl)phenyl]methyl}amino)-N,N-dimethylbutanamide

The above titled compound was prepared according to general sequence outlined in Scheme IV using 4-fluoro-2-(trifluoromethyl)benzonitrile (reagent A), [(4-trifluoromethylphenyl)methyl]amine (reagent B), 2-bromobutanoyl bromide (reagent C), and dimethylamine (reagent D). MS m/e 458.0 (M+H)⁺.

### Example 51

### 2-[[4-cyano-3-(trifluoromethyl)phenyl](phenylmethyl)amino]-N,N-dimethylbutanamide

The above titled compound was prepared according to general sequence outlined in Scheme IV using 4-fluoro-2-(trifluoromethyl)benzonitrile (reagent A), benzylamine (reagent B), 2-bromobutanoyl bromide (reagent C), and dimethylamine (reagent D). MS m/e 390.2 (M+H)⁺.

### Example 52

### N²-[4-cyano-3-(trifluoromethyl)phenyl]-N¹,N¹-dimethyl-N²-(2-thienylmethyl)alaninamide

The above titled compound was prepared according to general sequence outlined in Scheme IV using 4-fluoro-2-(trifluoromethyl)benzonitrile (reagent A), 2-thienylmethyl)amine (reagent B), 2-bromobutanoyl bromide (reagent C), and dimethylamine (reagent D). MS m/e 382.2 (M+H)⁺.

### Example 53

### 2-{{[3,4-bis(methyloxy)phenyl]methyl}[4-cyano-3-(trifluoromethyl)phenyl]amino}-N,N-dimethylbutanamide

The above titled compound was prepared according to general sequence outlined in Scheme IV using 4-fluoro-2-(trifluoromethyl)benzonitrile (reagent A), {[3,4-bis(methyloxy)phenyl]methyl}amine (reagent B), 2-bromobutanoyl bromide (reagent C), and dimethylamine (reagent D). MS m/e 450.4 (M+H)⁺.

### Example 54

### 2-{[4-cyano-3-(trifluoromethyl)phenyl][(3-methyl-2-thienyl)methyl]amino}-N,N-dimethylbutanamide

The above titled compound was prepared according to general sequence outlined in Scheme IV using 4-fluoro-2-(trifluoromethyl)benzonitrile (reagent A), [(3-methyl-2-thienyl)methyl]amine (reagent B), 2-bromobutanoyl bromide (reagent C), and dimethylamine (reagent D). MS m/e 410.2 (M+H)⁺.

### Example 55

### 2-{[4-cyano-3-(trifluoromethyl)phenyl][(5-methyl-2-furanyl)methyl]amino}-N,N-dimethylbutanamide

The above titled compound was prepared according to general sequence outlined in Scheme IV using 4-fluoro-2-(trifluoromethyl)benzonitrile (reagent A), [(5-methyl-2-furanyl)methyl]amine (reagent B), 2-bromobutanoyl bromide (reagent C), and dimethylamine (reagent D). MS m/e 394.0 (M+H)⁺.

### Example 56

### N²-(3-chloro-4-cyanophenyl)-N¹,N¹-dimethyl-N²-(2-phenylethyl)alaninamide

The above titled compound was prepared according to general sequence outlined in Scheme IV using 2-chloro-4-fluorobenzonitrile (reagent A), (2-phenylethyl)amine (reagent B), 2-bromopropionyl bromide (reagent C), and dimethylamine (reagent D). MS m/e 356.2 (M+H)^{+.}

### Example 57

### N²-(3-chloro-4-cyanophenyl)-N²-[2-(2-chlorophenyl)ethyl]-N¹,N¹-dimethylalaninamide

The above titled compound was prepared according to general sequence outlined in Scheme IV using 2-chloro-4-fluorobenzonitrile (reagent A), [2-(2-chlorophenyl)ethyl]amine (reagent B), 2-bromopropionyl bromide (reagent C), and dimethylamine (reagent D). MS m/e 390.0 (M+H)⁺.

### Example 58

### N²-(3-chloro-4-cyanophenyl)-N¹,N¹-dimethyl-N²-(2-thienylmethyl)norleucinamide

The above titled compound was prepared according to general sequence outlined in Scheme IV using 2-chloro-4-fluorobenzonitrile (reagent A), (2-thienylmethyl)amine (reagent B), 2-bromohexanoyl bromide (reagent C), and dimethylamine (reagent D). MS m/e 424.2 (M+H)^{+.}

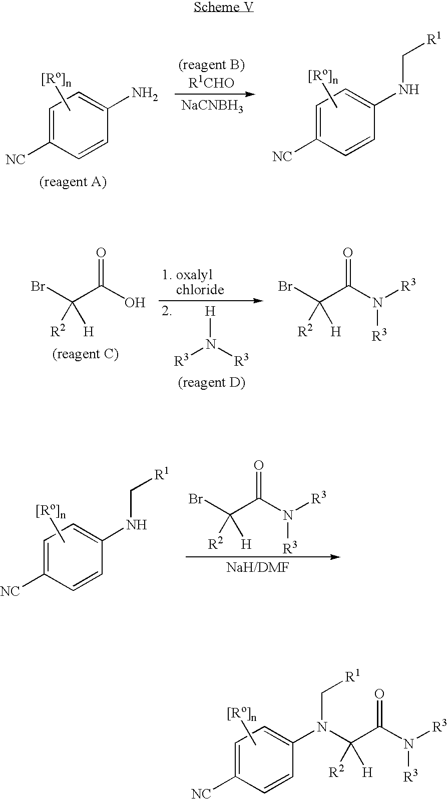

Examples 59 and 60 were prepared using the sequence outlined in Scheme V.

### Example 59

### N²-[4-cyano-3-(trifluoromethyl)phenyl]-N¹,N¹-dimethyl-N²-(2,2,2-trifluoroethyl)norvalinamide

### A. 4-[(2,2,2-trifluoroethyl)amino]-2-(trifluoromethyl)benzonitrile

To a slurry of 4-amino-2-(trifluoromethyl)benzonitrile (reagent A) (30.09 g, 162 mmol) and sodium cyanoborohydride (21.35 g, 340 mmol) in dichloromethane (160 mL) at ice bath temperature was added trifluoroacetic acid (160 mL, 2.08 mol). Trifluoroacetaldehyde hydrate (reagent B) (52.2 g, 405 mmol) was then added over 5 min. The mixture was stirred 10 min and warmed to room temperature. After 41 hours, the mixture was slowly poured into saturated sodium bicarbonate aqueous solution. Organic and aqueous phases of the filtrate were separated, and the aqueous layer extracted three times with dichloromethane. Combined organic extracts were concentrated to dryness. The oil residues were then purified by silica chromatography yielded the title compound as slightly tan plates, mp 132.5-134° C. ¹H NMR (300 MHz, MeOH-d₄) δ 7.59 (d, 1H), 7.05 (d, 1H), 6.92 (dd, 1H), 3.92 (q, 2H). MS m/z 268 (M⁺)

### B. 2-bromo-N,N-dimethylpentanamide

To a solution of 2-bromopentanoic acid (reagent C) (4.0 g, 22.1 mmol) in 30 mL dichloromethane was slowly added oxalyl chloride (4.2 gram, 33.1 mmol). The solution was refluxed two hours then cooled to room temperature. The solvent was removed and the residue was redissolved with 20 mL of dichloromethane. Dimethylamine (reagent D) (22 mL of 1.0 M THF solution, 44.2 mmol) was added and the mixture was stirred 30 min at room temperature. Aqueous 1N HCl was added to the reaction mixture, whereupon the organic portion was extracted using ethyl acetate. The organic layer was washed twice with saturated sodium bicarbonate and once with brine then dried over sodium sulfate. The oily residue was used in the next step without further purification.

C. N²-[4-cyano-3-(trifluoromethyl)phenyl]-N¹,N¹-dimethyl-N²-(2,2,2-trifluoroethyl)norvalinamide

To an ice cooled solution of 4-[(2,2,2-trifluoroethyl)amino]-2-(trifluoromethyl) benzonitrile (Step A) (250 mg, 0.94 mmol) in DMF (5 mL) was added sodium hydride (45 mg, 1.88 mmol). After stirring at 0° C. for 20 min, 2-bromo-N,N-dimethylpentanamide (Step B) (293 mg, 1.41 mmol) was added. The reaction was warmed to room temperature and continued to stir at this temperature for two hours. The reaction mixture was poured into water and the solution was extracted two times with diethyl ether. The organic layer was dried over sodium sulfate and concentrated. The oily residue was purified by silica gel chromatography. ¹H NMR (300 MHz, CDCl₃-d) δ 7.72 (d, 1H), 7.18 (d, 1H), 7.05 (dd, 1H), 4.61 (m, 1H), 4.06 (m, 2H), 2.98 (s, 6H), 2.03 (m, 1H), 1.59 (m, 1H), 1.35 (m, 2H), 0.97 (t, 3H); MS m/z 395 (M⁺)

### Example 60

### N²-[4-cyano-3-(trifluoromethyl)phenyl]-N¹N¹-dimethyl-N²-(2,2,2-trifluoroethyl)norleucinamide

The above titled compound was prepared according to general sequence outlined in Scheme V using 4-amino-2-(trifluoromethyl)benzonitrile (reagent A), trifluoroacetaldehyde hydrate (reagent B), 2-bromohexanoic acid (reagent C), and dimethylamine (reagent D). MS m/z 409 (M⁺)

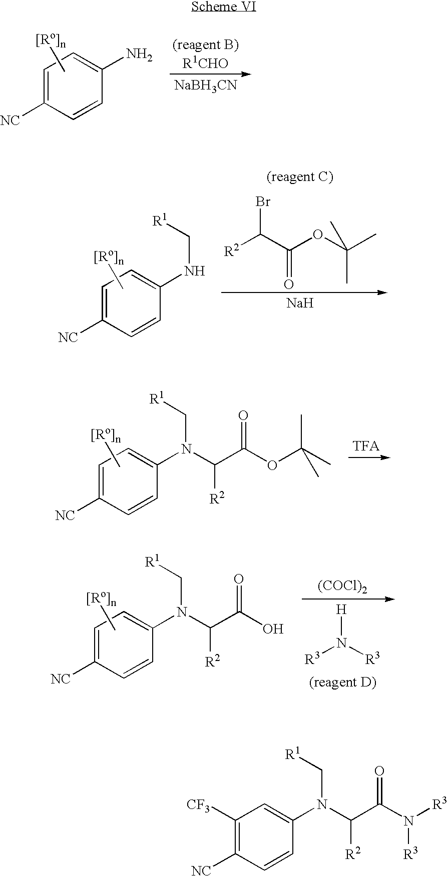

Examples 61-111 were prepared using the sequence outlined in Scheme VI.

### Example 61

### N²-[4-cyano-3-(trifluoromethyl)phenyl]-N¹,N¹-bis(2-methylpropyl)-N²-(2,2,2-trifluoroethyl)alaninamide

### A. 4-[(2,2,2-trifluoroethyl)amino]-2-(trifluoromethyl)benzonitrile

To a slurry of 4-amino-2-(trifluoromethyl)benzonitrile (reagent A) (30.09 g, 162 mmol) and sodium cyanoborohydride (21.35 g, 340 mmol) in dichloromethane (160 mL) at ice bath temperature was added trifluoroacetic acid (160 mL, 2.08 mol). Trifluoroacetaldehyde hydrate (reagent B) (52.2 g, 405 mmol) was then added over 5 min. The mixture was stirred 10 min and warmed to room temperature. After 41 hours, the mixture was slowly poured into saturated sodium bicarbonate aqueous solution. Organic and aqueous phases of the filtrate were separated, and the aqueous layer extracted three times with dichloromethane. Combined organic extracts were concentrated to dryness. The oil residues were then purified by silica chromatography yielded the title compound as slightly tan plates, mp 132.5-134° C. ¹H NMR (300 MHz, MeOH-d₄) δ 7.59 (d, 1H), 7.05 (d, 1H), 6.92 (dd, 1H), 3.92 (q, 2H). MS m/z 268 (M⁺)

### B. 1,1-Dimethylethyl-N-[4-cyano-3-(trifluoromethyl)phenyl]-N-(2,2,2-trifluoroethyl)alaninate

To an ice cooled solution of 4-(2,2,2-trifluoroethylamino)-2-trifluoromethyl-benzonitrile (1.5 g; 5.6 mmol) in DMF (30 mL) was added sodium hydride (269 mg; 11.2 mmol). After stirred at 0° C. for 20 min, 1,1-dimethylethyl 2-bromopropanoate (reagent C) (2.9 g, 14.1 mmol) was added. The reaction mixture was warmed to room temperature and continued to stir at this temperature for one hour. The reaction mixture was poured into water and the solution was extracted three times with diethyl ether. The organic layer dried over sodium sulfate and concentrated down. The oil residues were then purified by silica chromatography.

### C. N-[4-cyano-3-(trifluoromethyl)phenyl]-N-(2,2,2-trifluoroethyl)alanine

1,1-dimethylethyl-N-[4-cyano-3-(trifluoromethyl)phenyl]-N-(2,2,2-trifluoroethyl)alaninate (1 g, 2.53 mmol) was dissolved in dichloromethane (5 mL), and to the solution was added triethylsilane (2 mL) followed by trifluoroacetic acid (2 mL). The resulting solution was stirred 17 hours at room temperature and concentrated in vacuo. The residue was dissolved in 1N NaOH and washed three times with diethyl ether. The aqueous layer was acidified to pH 1 by 1N HCl (aq) and extracted three times with ethyl acetate. The combined organics were dried over Na₂SO₄ and concentrated to dryness. The crude product was not purified before the next step.

### D. N²-[4-cyano-3-(trifluoromethyl)phenyl]-N¹,N¹-bis(2-methylpropyl)-N²-(2,2,2-trifluoroethyl)alaninamide

To a slurry of N-[4-cyano-3-(trifluoromethyl)phenyl]-N-(2,2,2-trifluoroethyl)alanine (1.0 g, 2.94 mmol) in anhydrous dichloromethane (20 mL) was added oxalyl chloride (933 mg, 7.35 mmol) over 5 min. The mixture was brought to reflux under nitrogen. After 3 hours, the solution was cooled, and the solvent was evaporated. The residue was redissolved with 10 mL of dichloromethane. Diisobutylamine (reagent D) (1.90 g, 14.7 mmol) was added and the reaction was stirred at room temperature for one hour. The solvent was then removed and the crude product was purified by silica gel chromatography. ¹H NMR (300 MHz, CDCl₃-d) δ 7.68 (d, 1H), 7.18 (s, 1H), 7.06 (d, 1H), 4.85 (m, 1H), 4.20 (m, 2H), 3.20 (m, 4H), 1.97 (m, 2H), 1.55 (d, 3H), 0.90 (m, 12H). MS m/z 452 (M⁺+1)

### Example 62

### N¹,N¹-dibutyl-N²-[4-cyano-3-(trifluoromethyl)phenyl]-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and dibutylamine (reagent D). MS m/z 452 (M⁺+1)

### Example 63

### N²-[4-cyano-3-(trifluoromethyl)phenyl]-N¹-(1,1-dimethylethyl)-N¹-methyl-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and N,2-dimethyl-2-propanamine (reagent D). MS m/z 410 (M⁺+1)

### Example 64

### N²-[4-cyano-3-(trifluoromethyl)phenyl]-N¹-methyl-N¹-2-propyn-1-yl-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and N-methylpropargylamine (reagent D). MS m/z 392 (M⁺+1)

### Example 65

### N²-[4-cyano-3-(trifluoromethyl)phenyl]-N¹-ethyl-N¹-propyl-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and N-ethylpropylamine (reagent D). MS m/z 410 (M⁺+1)

### Example 66

### N²-[4-cyano-3-(trifluoromethyl)phenyl]-N¹-ethyl-N¹-(1-methylethyl)-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and N-ethylisopropylamine (reagent D). MS m/z 410 (M⁺+1)

### Example 67

### N²-[4-cyano-3-(trifluoromethyl)phenyl]-N¹-ethyl-N¹-methyl-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and N-ethylmethylamine (reagent D). MS m/z 382 (M⁺+1)

### Example 68

### N²-[4-cyano-3-(trifluoromethyl)phenyl]-N¹,N¹-dipropyl-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and dipropylamine (reagent D). MS m/z 424 (M⁺+1)

### Example 69

### N²-[4-cyano-3-(trifluoromethyl)phenyl]-N¹,N¹-diethyl-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and diethylamine (reagent D). MS m/z 396 (M⁺+1)

### Example 70

### 4-[[1-methyl-2-oxo-2-(1-piperidinyl)ethyl](2,2,2-trifluoroethyl)amino]-2-(trifluoromethyl)benzonitrile

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and piperidine (reagent D). MS m/z 408 (M⁺+1)

### Example 71

### 4-[[2-(1-azetidinyl)-1-methyl-2-oxoethyl](2,2,2-trifluoroethyl)amino]-2-(trifluoromethyl)benzonitrile

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and azetidine (reagent D). MS m/z 380 (M⁺+1)

### Example 72

### N²-[4-cyano-3-(trifluoromethyl)phenyl]-N¹-methyl-N¹-propyl-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and N-methyl-N-propylamine (reagent D). MS m/z 396 (M⁺+1)

### Example 73

### N¹-butyl-N¹-(cyanomethyl)-N²-[4-cyano-3-(trifluoromethyl)phenyl]-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and (butylamino)acetonitrile (reagent D). MS m/z 435 (M⁺+1)

### Example 74

### N²-[4-cyano-3-(trifluoromethyl)phenyl]-N¹-[2-(methyloxy)ethyl]-N¹-propyl-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and N-(2-methoxyethyl)-N-propylamine (reagent D). MS m/z 440 (M⁺+1)

### Example 75

### N¹,N¹-bis(cyanomethyl)-N²-[4-cyano-3-(trifluoromethyl)phenyl]-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and 2,2′-iminodiacetonitrile (reagent D). MS m/z 418 (M⁺+1)

### Example 76

### 4-[[-1-methyl-2-(4-morpholinyl)-2-oxoethyl](2,2,2-trifluoroethyl)amino]-2-(trifluoromethyl)benzonitrile

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and morpholine (reagent D). MS m/z 410 (M⁺+1)

### Example 77

### N²-[4-cyano-3-(trifluoromethyl)phenyl]-N¹-(cyclopropylmethyl)-N¹-propyl-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and (cyclopropylmethyl)propylamine (reagent D). MS m/z 436 (M⁺+1)

### Example 78

### N²-[4-cyano-3-(trifluoromethyl)phenyl]-N¹-cyclohexyl-N¹-methyl-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and N-cyclohexylmethylamine (reagent D). MS m/z 436 (M⁺+1)

### Example 79

### N²-[4-cyano-3-(trifluoromethyl)phenyl]-N¹-methyl-N¹-[2-(methyloxy)ethyl]-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and N-(2-methoxyethyl)methylamine (reagent D). MS m/z 412 (M⁺+1)

### Example 80

### 4-[[1-methyl-2-oxo-2-(4thiomorpholinyl)ethyl](2,2,2-trifluoroethyl)amino]-2-(trifluoromethyl)benzonitrile

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and thiomorpholine (reagent D). MS m/z 426 (M⁺+1)

### Example 81

### N¹-(2-cyanoethyl)-N²-[4-cyano-3-(trifluoromethyl)phenyl]-N¹-methyl-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and 3-(methylamino)propanenitrile (reagent D). MS m/z 407 (M⁺+1)

### Example 82

### 4-[[1-methyl-2-oxo-2-(1-pyrrolidinyl)ethyl](2,2,2-trifluoroethyl)amino]-2-(trifluoromethyl)benzonitrile

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and pyrrolidine (reagent D). MS m/z 394 (M⁺+1)

### Example 83

### N²-[4-cyano-3-(trifluoromethyl)phenyl]-N¹-methyl-N¹-(1-methylethyl)-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and N-methylisopropylamine (reagent D). MS m/z 396 (M⁺+1)

### Example 84

### N²-[4-cyano-3-(trifluoromethyl)phenyl]-N¹,N¹-di-2-propen-1-yl-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and di-2-propen-1-ylamine (reagent D). MS 7m/z 420 (M⁺+1)

### Example 85

### N²-[4cyano-3-(trifluoromethyl)phenyl]-N¹-methyl-N¹-2-propen-1-yl-N²-(2,2,2-trifluoroethyl)alaninamide

The above titled compound was prepared according to general sequence outlined in Scheme VI using 4-amino-2-(trifluoromethyl)benzonitrile (reagent A), trifluoroacetaldehyde hydrate (reagent B), 2-bromopropionic acid (reagent C), and N-methylallylamine (reagent D). MS m/z 394 (M⁺+1)

### Example 86

### N,N-dibutyl-2-[[4-cyano-3-(trifluoromethyl)phenyl](2,2,2-trifluoroethyl)amino]butanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and dibutylamine (reagent D). MS m/z 466 (M⁺+1)

### Example 87

### 2-[[4-cyano-3-(trifluoromethyl)phenyl](2,2,2-trifluoroethyl)amino]-N-(1,1-dimethylethyl)-N-methylbutanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and N,2-dimethyl-2-propanamine (reagent D). MS m/z 424 (M⁺+1)

### Example 88

### 2-[[4-cyano-3-(trifluoromethyl)phenyl](2,2,2-trifluoroethyl)amino]-N-methyl-N-2-propyn-1-ylbutanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and (methylamine)acetonitrile (reagent D). MS m/z 405 (M⁺+1)

### Example 89

### 2-[[4-cyano-3-(trifluoromethyl)phenyl](2,2,2-trifluoroethyl)amino]-N-ethyl-N-propylbutanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and N-ethylpropylamine (reagent D). MS m/z 424 (M⁺+1)

### Example 90

### 2-[[4-cyano-3-(trifluoromethyl)phenyl](2,2,2-trifluoroethyl)amino]-N-ethyl-N-(1-methylethyl)butanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and N-ethylisopropylamine (reagent D). MS m/z 424 (M⁺+1)

### Example 91

### 2-[[4-cyano-3-(trifluoromethyl)phenyl](2,2,2-trifluoroethyl)amino]-N,N-bis(2-methylpropyl)butanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and diisobutylamine (reagent D). MS m/z 466 (M⁺+1)

### Example 92

### 2-[[4-cyano-3-(trifluoromethyl)phenyl](2,2,2-trifluoroethyl)amino]-N-ethyl-N-methylbutanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and N-ethylmethylamine (reagent D). MS m/z 396 (M⁺+1)

### Example 93

### 2-[[4-cyano-3-(trifluoromethyl)phenyl](2,2,2-trifluoroethyl)amino]-N,N-dipropylbutanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and dipropylamine (reagent D). MS m/z 438 (M⁺+1)

### Example 94

### 2-[[4-cyano-3-(trifluoromethyl)phenyl](2,2,2-trifluoroethyl)amino]-N,N-diethylbutanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and diethylamine (reagent D). MS m/z 410 (M⁺+1)

### Example 95

### 4-[[1-(1-piperidinylcarbonyl)propyl](2,2,2-trifluoroethyl)amino]-2-(trifluoromethyl)benzonitrile

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and piperidine (reagent D). MS m/z 422 (M⁺+1)

### Example 96

### 4-[[1-(1-azetidinylcarbonyl)propyl](2,2,2-trifluoroethyl)amino]-2-(trifluoromethyl)-benzonitrile

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and azetidine (reagent D). MS m/z 394 (M⁺+1)

### Example 97

### 2-[[4-cyano-3-(trifluoromethyl)phenyl](2,2,2-trifluoroethyl)amino]-N-methyl-N-propylbutanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and N-methylpropylamine. (reagent D). MS m/z 410 (M⁺+1)

### Example 98

### N-butyl-N-(cyanomethyl)-2-[[4-cyano-3-(trifluoromethyl)phenyl](2,2,2-trifluoroethyl)amino]butanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and N-butylaminoacetonitrile (reagent D). MS m/z 449 (M⁺+1)

### Example 99

### 2-[[4-cyano-3-(trifluoromethyl)phenyl](2,2,2-trifluoroethyl)amino]-N-[2-(methyloxy)ethyl]-N-propylbutanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and N-(2-methoxyethyl)-N-propylamine (reagent D). MS m/z 454 (M⁺+1)

### Example 100

### N,N-bis(cyanomethyl)-2-[[4-cyano-3-(trifluoromethyl)phenyl](2,2,2-trifluoroethyl)amino]butanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and 2,2′-iminodiacetonitrile (reagent D). MS m/z 432 (M⁺+1)

### Example 101

### 4-[[1-(4-morpholinylcarbonyl)propyl](2,2,2-trifluoroethyl)amino]-2-(trifluoromethyl)benzonitrile

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and morpholine (reagent D). MS m/z 424 (M⁺+1)

### Example 102

### 2-[[4-cyano-3-(trifluoromethyl)phenyl](2,2,2-trifluoroethyl)amino]-N-(cyclopropylmethyl)-N-propylbutanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and (cyclopropylmethyl)methylamine (reagent D). MS m/z 450 (M⁺+1)

### Example 103

### 2-[[4-cyano-3-(trifluoromethyl)phenyl](2,2,2-trifluoroethyl)amino]-N-cyclohexyl-N-methylbutanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and N-methylcyclohexylamine (reagent D). MS m/z 450 (M⁺+1)

### Example 104

### 4-[[1-(4-thiomorpholinylcarbonyl)propyl](2,2,2-trifluoroethyl)amino]-2-(trifluoromethyl)benzonitrile

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and thiomorpholine (reagent D). MS m/z 440 (M⁺+1)

### Example 105

### 2-[[4-cyano-3-(trifluoromethyl)phenyl](2,2,2-trifluoroethyl)amino]-N-methyl-N-[2-(methyloxy)ethyl]butanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and N-(2-methoxyethyl)methylamine (reagent D). MS m/z 426 (M⁺+1)

### Example 106

### N-(2-cyanoethyl)-2-[[4-cyano-3-(trifluoromethyl)phenyl](2,2,2-trifluoroethyl)-amino]-N-methylbutanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and 3-(methylamino)propanenitrile (reagent D). MS m/z 421 (M⁺+1)

### Example 107

### 4-[[1-(1-pyrrolidinylcarbonyl)propyl](2,2,2-trifluoroethyl)amino]-2-(trifluoromethyl)benzonitrile

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and pyrrolidine (reagent D). MS m/z 408 (M⁺+1)

### Example 108

### 2-[[4-cyano-3-(trifluoromethyl)phenyl](2,2,2-trifluoroethyl)amino]-N-methyl-N-(1-methylethyl)butanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and N-methylisopropylamine (reagent D). MS m/z 410 (M⁺+1)

### Example 109

### 2-[[4-cyano-3-(trifluoromethyl)phenyl](2,2,2-trifluoroethyl)amino]-N,N-di-2-propen-1-ylbutanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and di-2-propen-1-ylamine (reagent D). MS m/z 434 (M⁺+1)

### Example 110

### 4-[[1-(1,3-thiazolidin-3-ylcarbonyl)propyl](2,2,2-trifluoroethyl)amino]-2-(trifluoromethyl)benzonitrile

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and thiazolidine (reagent D). MS m/z 426 (M⁺+1)

### Example 111

### 2-[[4-cyano-3-(trifluoromethyl)phenyl](2,2,2-trifluoroethyl)amino]-N-methyl-N-2-propen-1-ylbutanamide

The above titled compound was prepared according to general sequence outlined in Scheme VI using 4-amino-2-(trifluoromethyl)benzonitrile (reagent A), trifluoroacetaldehyde hydrate (reagent B), 2-bromobutanoic acid (reagent C), and N-methyl-allylamine (reagent D). MS m/z 408 (M⁺+1)

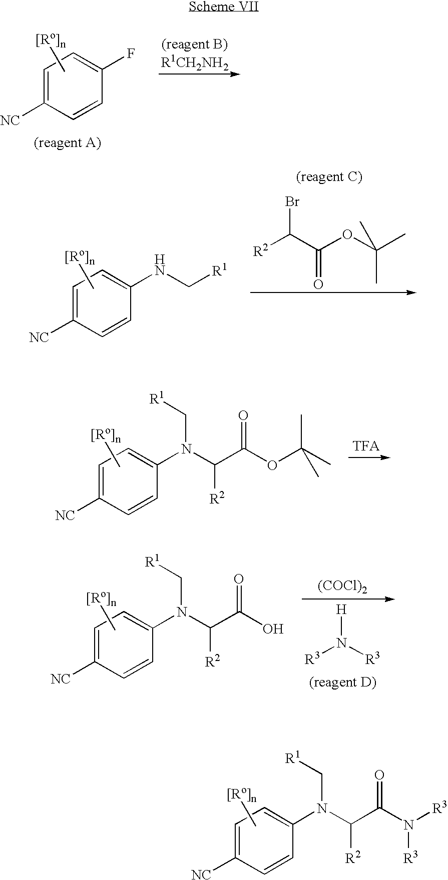

Examples 112-117 were prepared using the sequence outlined in Scheme VII.

### Example 112

### N²-[4-cyano-3-(trifluoromethyl)phenyl]-N¹,N¹-dimethyl-N²-{[2-(trifluoromethyl)-phenyl]methyl}alaninamide

### A. 2-(trifluoromethyl)-4-({[2-(trifluoromethyl)phenyl]methyl}amino)benzonitrile

Freshly oven dried potassium carbonate (5.5 g, 39.75 mmol) was added to the 50 mL DMF solution of 4-fluoro-2-(trifluoromethyl)benzonitrile (reagent A) (5 g, 26.5 mmol) and 2-trifluoromethylbenzylamine (reagent B) (5.6 g, 31.7 mmol). The reaction was heated at 95° C. for two hours before cooling to room temperature. The reaction was diluted with water then extracted with diethyl ether three times. The organic layer was dried over Na₂SO₄, then purified by silica chromatography.

### B. 1,1-dimethylethyl N-[4-cyano-3-(trifluoromethyl)phenyl]-N-{[2-(trifluoromethyl)phenyl]methyl}alaninate

The title compound was prepared according to the procedure given in Example 61, part B using 2-(trifluoromethyl)-4-({[2-(trifluoromethyl)phenyl]methyl}amino)benzonitrile and 1,1-dimethylethyl 2-bromopropionate (reagent C).

### C. N-[4-cyano-3-(trifluoromethyl)phenyl]-N-{[2-(trifluoromethyl)phenyl]methyl}alanine

The title compound was prepared according to the procedure given in Example 61, part C starting with N-[4-cyano-3-(trifluoromethyl)phenyl]-N-{[2-(trifluoromethyl)phenyl]methyl}alanine.

### D. N²-[4-cyano-3-(trifluoromethyl)phenyl]-N¹,N¹-dimethyl-N²-{[2-(trifluoromethyl)-phenyl]methyl}alaninamide

The title compound was prepared according to the procedure given in Example 61, part D using N-[4-cyano-3-(trifluoromethyl)phenyl]-N-{[2-(trifluoromethyl)phenyl]methyl}alanine and dimethylamine (reagent D). ¹H NMR (300 MHz, CDCl₃-d) δ 7.72 (d, 1H), 7.55 (d, 1H), 7.45 (m, 1H), 7.37 (m, 1H), 7.25 (m, 1H), 6.85 (s, 1H), 6.66 (m, 1H), 5.05 (m, 1H), 4.91 (m, 2H), 3.10 (s, 3H), 2.98 (s, 3H), 1.49 (d, 3H). MS m/z 444 (M⁺+1)

### Example 113

### N²-[4-cyano-3-(trifluoromethyl)phenyl]-N²-(2,2-dimethylpropyl)-N¹,N¹-dimethylalaninamide

The above titled compound was prepared according to general sequence outlined in Scheme VII using 4-fluoro-2-(trifluoromethyl)benzonitrile (reagent A), 2,2-dimethyl-1-propanamide (reagent B), 1,1-dimethylethyl 2-bromopropionate (reagent C), and dimethylamine (reagent D). MS m/z 356 (M⁺+1)

### Example 114

### N²-[4-cyano-3-(trifluoromethyl)phenyl]-N²-(3,3-dimethylbutyl)-N¹,N¹-dimethylalaninamide

The above titled compound was prepared according to general sequence outlined in Scheme VII using 4-fluoro-2-(trifluoromethyl)benzonitrile (reagent A), 3,3-dimethyl-1-butanamine (reagent B), 1,1-dimethylethyl 2-bromopropionate (reagent C), and dimethylamine (reagent D). MS m/z 370 (M⁺+1)

### Example 115

### N²-[4-cyano-3-(trifluoromethyl)phenyl]-N¹,N¹-dimethyl-N²-(3,3,3-trifluoropropyl)-alaninamide

The above titled compound was prepared according to general sequence outlined in Scheme VII using 4-fluoro-2-(trifluoromethyl)benzonitrile (reagent A), 3,3,3-trifluoropropanamine (reagent B), 1,1-dimethylethyl 2-bromopropionate (reagent C), and dimethylamine (reagent D). MS m/z 382 (M⁺+1)

### Example 116

### N²-[2,2-bis(methyloxy)ethyl]-N²-[4-cyano-3-(trifluoromethyl)phenyl]-N¹,N¹-dimethylalaninamide

The above titled compound was prepared according to general sequence outlined in Scheme VII using 4-fluoro-2-(trifluoromethyl)benzonitrile (reagent A), 2,2-bis(methyloxy)ethanamine (reagent B), 1,1-dimethylethyl 2-bromopropionate (reagent C), and dimethylamine (reagent D). MS m/z 373 (M⁺+22)

### Example 117

### 2-[[4-cyano-3-(trifluoromethyl)phenyl](2,2-dimethylpropyl)amino]-N,N-dimethylbutanamide

The above titled compound was prepared according to general sequence outlined in Scheme VII using 4-fluoro-2-(trifluoromethyl)benzonitrile (reagent A), 2,2-dimethylpropanamine (reagent B), 1,1-dimethylethyl 2-bromohexanoate (reagent C), and dimethylamine (reagent D). MS m/z 370 (M⁺+1)

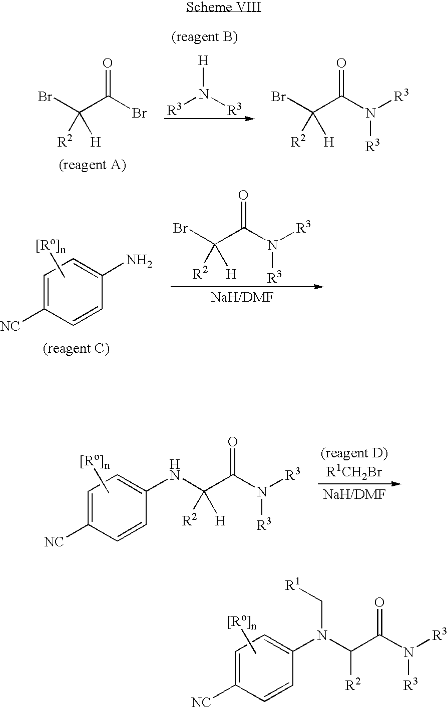

Examples 118-122 were prepared using the sequence outlined in Scheme VIIsI.

### Example 118

### N²-[(2-chlorophenyl)methyl]-N²-(4-cyanophenyl)-N¹,N¹-dimethylalaninamide

### A. 2-Bromo-N,N-dimethylpropanamide

To a solution of dimethyl amine (reagent B) (2.0M in THF, 30 mL) was added 2-bromopropanoyl bromide (reagent A) (8.7 g, 0.04 mmol) at 0° C. The resulting mixture was kept stirring at 0° C. for additional 10 mins. After the reaction was filtered the filtrate was washed with very dilute HCl and dried over sodium sulfate. Solvent was removed to give 5.12 g (70% yield) of the title compound as a colorless oil MS m/e 180.0 (M+H)⁺. The title compound was carried over to the next step without further purification.

### B. N²-(4-Cyanophenyl)-N¹,N¹-dimethylalaninamide

4-Aminobenzonitrile (0.0673 g, 0.57 mmol) (reagent C) was added to a suspension of NaH (60% in mineral oil, 0.16 g, 3.9 mmol) in DMF (3 mL) at 0° C. After stirring for 5 min, 2-bromo-N,N-dimethylpropanamide (Step A) (0.112 g, 0.62 mmol) was added and the reaction mixture was stirred at 55° C. for 3 h. To this crude reaction mixture was added 1-(bromomethyl)-2-chlorobenzene (reagent D) (0.127 g, 0.62 mmol) and the reaction mixture was stirred at 55° C. for additional 3 h. (In some circumstances, heating at 60-120° C. was necessary to progress a slow reaction). After cooling to room temperature, excess sodium hydride was quenched with saturated NH₄Cl, and the reaction mixture was extracted with EtOAc. The organic layer was dried over Na₂SO₄, concentrated and purified via preparative HPLC (XTerra PreP RP, 19×150 mm, 19 mL/min, A: acetonitrile B: water, A: 25 to 95% over 15 min, UV detection at 214 nm) to give the title compound as a light yellow solid (19 mg, 10%). ¹H NMR (400 MHz, CDCl₃) δ 7.461 (m, 2H), 7.413 (dd, 1H, J=1.6 Hz, 7.6 Hz), 7.193 (m, 2H), 7.073 (m, 1H), 6.620 (dd, 2H, J=2 Hz, 7.2 Hz), 4.910 (m, 1H), 4.755 (m, 2H), 3.101 (s, 3H), 2.910 (s, 3H), 1.479 (d, 3H, J=6.8 Hz). MS m/e 342.2 (M+H)⁺.

### Example 119

### N²-[4-cyano-3-(trifluoromethyl)phenyl]-N²-(2,2-dimethylpropyl)-N¹,N¹-dimethylnorleucinamide

The above titled compound was prepared according to general sequence outlined in Scheme VIII using 2-bromopropionyl bromide (reagent A), dimethylamine (reagent B), 4-amino-2-trifluoromethylbenzonitrile (reagent C), and 1-bromo-2,2-dimethylpropane (reagent D). MS m/e 398.2 (M+H)⁺.

### Example 120

### N²-(3-chloro-4-cyanophenyl)-N²-ethyl-N¹,N¹-dimethylalaninamide

The above titled compound was prepared according to general sequence outlined in Scheme VIII using 2-bromopropionyl bromide (reagent A), dimethylamine (reagent B), 4-fluoro-2-chlorobenzonitrile (reagent C), and iodoethane (reagent D). MS m/e 280.2 (M+H)^{+.}

### Example 121

### N²-(3-chloro-4-cyanophenyl)-N²-[(2-chlorophenyl)methyl]-N¹,N¹-dimethylalaninamide

The above titled compound was prepared according to general sequence outlined in Scheme VIII using 2-bromopropionyl bromide (reagent A), dimethylamine (reagent B), 4-fluoro-2-chlorobenzonitrile (reagent C), and [2-(chloromethyl)phenyl]methylbromide (reagent D). ¹H NMR (400 MHz, CDCl₃) δ 7.435 (m, 2H), 7.218 (m, 2H), 7.033 (m, 1H), 6.668 (d, 1H, J=2.8 Hz), 6.491 (dd, 1H, J=2.8 Hz, 8.8 Hz), 4.863 (m, 1H), 4.761 (s, 2H), 3.114 (s, 3H), 1.484 (d, 3H, J=7.2 Hz).

### Example 122

### Preparation of N²-[4-cyano-3-(trifluoromethyl)phenyl]-N²-ethyl-N¹,N¹,2-trimethylalaninamide

The above titled compound was prepared according to general sequence outlined in Scheme VIII using 2-bromo-2-methylpropionyl bromide (reagent A), dimethylamine (reagent B), 4-fluoro-2-trifluoromethylbenzonitrile (reagent C), and iodoethane (reagent D). MS m/e 328.4 (M+H)⁺

1. A compound represented by the following formula: 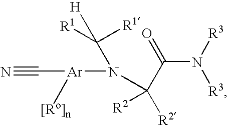
or a pharmaceutically acceptable salt thereof, or a pharmaceutically acceptable solvate thereof, or a combination thereof;
wherein n is 0, 1, or 2;
Ar is phenyl or naphthyl;
each R^{o} is independently CF₃, halo, C₁₋₆-alkyl, CN, or substituted C₁₋₆-alkyl;
R¹ and R^{1′} are each independently H, C₁₋₆-alkyl, substituted C₁₋₆-alkyl, C₂₋₆-alkenyl, C₃₋₆-cycloalkyl, substituted C₃₋₆-cycloalkyl, aryl, substituted aryl, heteroaryl, or substituted heteroaryl, or together with the carbon atom to which they are both attached form a C₅₋₆-cycloalkyl or C₅₋₆-cycloalkenyl group;
R² and R^{2′} are each independently H, C₁₋₆-alkyl, substituted C₁₋₆-alkyl, C₃₋₆-cycloalkyl, substituted C₃₋₆-cycloalkyl, —(CH₂)ₘ—X—C₃₋₆-cycloalkyl, —(CH₂)ₘ—X-substituted C₃₋₆-cycloalkyl, —(CH₂)ₘ—X-phenyl, —(CH₂)ₘ—X-substituted phenyl, —(CH₂)ₘ—X-pyridyl, or —(CH₂)ₘ—X-substituted pyridyl; and X is a bond, —O— or —S—, where m is 0-4 when X is a bond and m is 1-4 when X is —O— or —S—; with the proviso that R² and R^{2′} are not both H; and
each R³ is independently C₁₋₆-alkyl, substituted C₁₋₆-alkyl, C₁₋₆-alkenyl, or propargyl, or together with the nitrogen to which they are attached form a 3-6-membered heterocycloalkyl group;
with the proviso that when a) Ar is phenyl; b) R² is methyl or ethyl; c) R^{2′} is H; d) each R³ is methyl; e) R¹ is H; f) R^{o} is CF₃ ortho to the CN, and g) n=1; then R^{1′} is not cyclopropyl or CF₃.

2. The compound of claim 1 which is represented by the following formula: 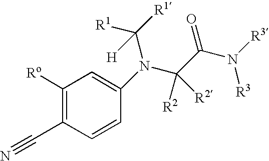
or a pharmaceutically acceptable salt thereof or a pharmaceutically acceptable solvate thereof or a combination thereof;
wherein R^{o} is CF₃, F, Cl, or methyl;
R¹ and R^{1′} are each independently H, methyl, ethyl, isopropyl, t-butyl, 2,2-dimethylpropyl, —CF₃, —CH₂CF₃, —CH(OCH₃)₂, —C(CH₃)═CH₂, phenyl, furyl, thienyl, trifluoromethylphenyl, chlorophenyl, methoxyphenyl, or tolyl, or together with the carbon to which they are attached form a cyclohexyl or cyclohexenyl group;
R² and R^{2′} are each independently H, methyl, n-propyl, isopropyl, n-butyl, sec-butyl, phenyl, benzyl, trifluoromethylphenyl, chlorophenyl, methoxyphenyl, or tolyl; and
Each R³ is independently methyl, ethyl, n-propyl, isopropyl, n-butyl, t-butyl, isobutyl, 2,2-dimethylbutyl, methoxymethyl, methoxyethyl, cyanomethyl, cyanoethyl, cyclopropylmethyl, 1-propenyl, or propargyl, or together with the nitrogen to which they are attached form an aziridinyl, propyleniminyl, pyrrolidinyl, piperidinyl, piperazinyl, thiazolidinyl, thiomorpholino, or morpholino group.

3. The compound of claim 2 which is represented by the following formula: 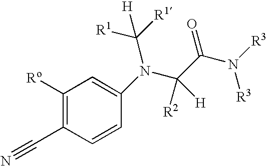
or a pharmaceutically acceptable salt thereof or a pharmaceutically acceptable solvate thereof or a combination thereof;
wherein R^{o} is CF₃ or Cl;
wherein R¹ is methyl, ethyl, isopropyl, t-butyl, —CH₂C(CH₃)₃, —CF₃, —CH₂CF₃, —CH(OCH₃)₂, isopropenyl, phenyl, furyl, thienyl, trifluoromethylphenyl, chlorophenyl, methoxyphenyl, or tolyl;
R^{1′} is H or methyl, or R¹ and R^{1′}, together with the carbon to which they are attached form a cyclohexyl or cyclohexenyl group;
R² is methyl, n-propyl, isopropyl, n-butyl, sec-butyl, phenyl, benzyl, trifluoromethylphenyl, chlorophenyl, methoxyphenyl, or tolyl; and
each R³ is independently methyl, ethyl, n-propyl, isopropyl, n-butyl, t-butyl, isobutyl, 2,2-dimethylbutyl, methoxymethyl, methoxyethyl, cyanomethyl, cyanoethyl, cyclopropylmethyl, 1-propenyl, or propargyl, or together with the nitrogen to which they are attached form a propyleniminyl, piperidinyl, pyrrolidinyl, thiomorphlino, thiazolidinyl, or morpholino group.

4. The compound of claim 1 or a pharmaceutically acceptable salt thereof or a pharmaceutically acceptable solvate thereof or a combination thereof which has an IC₅₀ of less than 10 μM.

5. A method of treating a patient comprising administering to the patient an effective amount of the compound of claim 1 or a pharmaceutically-acceptable salt thereof, or a solvate thereof, or combination thereof to treat endometreosis or uterine fibroids.

6. A composition comprising the compound of claim 3 and a pharmaceutically acceptable carrier therefor.

7. A compound selected from the group consisting of:
N²-[(2-chlorophenyl)methyl]-N²-[4-cyano-3-(trifluoromethyl)phenyl]-N¹,N¹-dimethyl-L-alaninamide;
N²-[4-cyano-3-(trifluoromethyl)phenyl]-N²-isobutyl-N¹,N¹-dimethyl-L-alaninamide;
N²-[4-cyano-3-(trifluoromethyl)phenyl]-N¹,N¹-dimethyl-N²-(1-phenylethyl)-L-alaninamide;
N²-[4-cyano-3-(trifluoromethyl)phenyl]-N¹,N¹-dimethyl-N²-(2-methyl-2-propen-1-yl)-L-isoleucinamide;
N²-[4-cyano-3-(trifluoromethyl)phenyl]-N¹,N¹-dimethyl-N²-(1-methylethyl)-L-alaninamide;
N²-[(2-chlorophenyl)methyl]-N²-[4-cyano-3-(trifluoromethyl)phenyl]-N¹,N¹-dimethyl-L-isoleucinamide;
N²-[4-cyano-3-(trifluoromethyl)phenyl]-N¹,N¹-dimethyl-N²-(2-methylpropyl)-L-isoleucinamide;
N²-[(2-chlorophenyl)methyl]-N²-[4-cyano-3-(trifluoromethyl)phenyl]-N¹,N¹-dimethyl-L-valinamide;
N²-[4-cyano-3-(trifluoromethyl)phenyl]-N¹,N¹-dimethyl-N²-{[2-(trifluoromethyl)phenyl]methyl}-L-valinamide;
N²-[4-cyano-3-(trifluoromethyl)phenyl]-N¹,N¹-dimethyl-N²-(2-methyl-2-propen-1-yl)-L-valinamide;
N²-[4-cyano-3-(trifluoromethyl)phenyl]-N¹,N¹-dimethyl-N²-{[2-(trifluoromethyl)phenyl]methyl}-L-isoleucinamide;
N²-[4-cyano-3-(trifluoromethyl)phenyl]-N¹,N¹-dimethyl-N²-(1-phenylethyl)-L-isoleucinamide;
N²-[4-cyano-3-(trifluoromethyl)phenyl]-N¹,N¹-dimethyl-N²-(2-methylpropyl)-L-valinamide;
N²-[(2-chlorophenyl)methyl]-N²-[4-cyano-3-(trifluoromethyl)phenyl]-N¹,N¹-dimethyl-D-alaninamide;
N²-[4-cyano-3-(trifluoromethyl)phenyl]-N²-2-cyclohexen-1-yl-N¹,N¹-dimethyl-L-alaninamide;
N²-[4-cyano-3-(trifluoromethyl)phenyl]-N²-cyclohexyl-N¹,N¹-dimethyl-L-alaninamide;
N²-[(2-chlorophenyl)methyl]-N²-[4-cyano-3-(trifluoromethyl)phenyl]-3-cyclohexyl-N¹,N¹-dimethyl-L-alaninamide;
2-{[(2-chlorophenyl)methyl][4-cyano-3-(trifluoromethyl)phenyl]amino}-N,N-dimethyl-2-phenylacetamide;
N²-[(2-chlorophenyl)methyl]-N²-[4-cyano-3-(trifluoromethyl)phenyl]-N¹,N¹-dimethyl-D-valinamide;
N-[(2-chlorophenyl)methyl]-N-[4-cyano-3-(trifluoromethyl)phenyl]-N,N,O-trimethyl-3-(methyloxy)-L-tyrosinamide;
N²-[(2-chlorophenyl)methyl]-N²-[4-cyano-3-(trifluoromethyl)phenyl]-N¹,N¹-dimethyl-3-(2-pyridinyl)alaninamide;
N²-[(2-chlorophenyl)methyl]-N²-[4-cyano-3-(trifluoromethyl)phenyl]-N¹,N¹-dimethyl-L-leucinamide;
N²-[(2-chlorophenyl)methyl]-N²-[4-cyano-3-(trifluoromethyl)phenyl]-N¹,N¹-dimethyl-D-leucinamide;
N²-[(2-chlorophenyl)methyl]-N²-[4-cyano-3-(trifluoromethyl)phenyl]-N¹,N¹-dimethyl-D-norleucinamide;
N²-[(2-chlorophenyl)methyl]-N²-[4-cyano-3-(trifluoromethyl)phenyl]-N¹,N¹-dimethyl-L-norleucinamide;
(2S)-2-{[(2-chlorophenyl)methyl][4-cyano-3-(trifluoromethyl)phenyl]amino}-N¹,N¹-dimethylbutanamide;
(2R)-2-{[(2-chlorophenyl)methyl][4-cyano-3-(trifluoromethyl)phenyl]amino}-N,N-dimethylbutanamide;
N²-[4-cyano-3-(trifluoromethyl)phenyl]-N²-(2,2-dimethylpropyl)-N¹,N¹-dimethyl-L-valinamide;
N²-[(2-chlorophenyl)methyl]-N²-(4-cyano-3-fluorophenyl)-N¹,N¹-dimethylalaninamide;
N²-[4-cyano-3-(trifluoromethyl)phenyl]-N¹,N¹-dimethyl-N²-{[2-(methyloxy)phenyl]methyl}alaninamide;
N²-[4-cyano-3-(trifluoromethyl)phenyl]-N¹,N¹-dimethyl-N²-[(2-methylphenyl)methyl]alaninamide;
N²-[4-cyano-3-(trifluoromethyl)phenyl]-N²-(2-furanylmethyl)-N¹,N¹-dimethylalaninamide;
N²-[4-cyano-3-(trifluoromethyl)phenyl]-N¹,N¹ ,N²-trimethylalaninamide;
N²-[4-cyano-3-(trifluoromethyl)phenyl]-N²-ethyl-N¹,N¹-dimethylalaninamide;
2-[[4-cyano-3-(trifluoromethyl)phenyl](methyl)amino]-N,N-dimethylbutanamide;
2-[[4-cyano-3-(trifluoromethyl)phenyl](ethyl)amino]-N,N-dimethylbutanamide;
N²-[4-cyano-3-(trifluoromethyl)phenyl]-N²-(2-furanylmethyl)-N¹,N¹-dimethylnorleucinamide;
2-[[4-cyano-3-(trifluoromethyl)phenyl](2-thienylmethyl)amino]-N,N-dimethylbutanamide;
2-[[4-cyano-3-(trifluoromethyl)phenyl](2-furanylmethyl)amino]-N,N-dimethylbutanamide;
2-{[(2-chlorophenyl)methyl][4-cyano-3-(trifluoromethyl)phenyl]amino}-N,N-dimethylbutanamide;
2-[[4-cyano-3-(trifluoromethyl)phenyl](2-pyridinylmethyl)amino]-N,N-dimethylbutanamide;
N²-[4-cyano-3-(trifluoromethyl)phenyl]-N²-[(2-fluorophenyl)methyl]-N¹,N¹-dimethylalaninamide;
N²-[4-cyano-3-(trifluoromethyl)phenyl]-N¹,N¹-dimethyl-N²-{[3-(trifluoromethyl)phenyl]methyl}alaninamide;
N²-[4-cyano-3-(trifluoromethyl)phenyl]-N¹,N¹-dimethyl-N²-{[4-(trifluoromethyl)phenyl]methyl}alaninamide;
N²-[4-cyano-3-(trifluoromethyl)phenyl]-N¹,N¹-dimethyl-N²-(phenylmethyl)alaninamide;
2-([4-cyano-3-(trifluoromethyl)phenyl]{[2-(methyloxy)phenyl]methyl}amino)-N,N-dimethylbutanamide;
2-{[4-cyano-3-(trifluoromethyl)phenyl][(2-methylphenyl)methyl]amino}-N,N-dimethylbutanamide;
2-{[4-cyano-3-(trifluoromethyl)phenyl][(2-fluorophenyl)methyl]amino}-N,N-dimethylbutanamide;
2-([4-cyano-3-(trifluoromethyl)phenyl]{[3-(trifluoromethyl)phenyl]methyl}amino)-N,N-dimethylbutanamide;
2-([4-cyano-3-(trifluoromethyl)phenyl]{[4-(trifluoromethyl)phenyl]methyl}amino)-N,N-dimethylbutanamide;
2-[[4-cyano-3-(trifluoromethyl)phenyl](phenylmethyl)amino]-N,N-dimethylbutanamide;
N²-[4-cyano-3-(trifluoromethyl)phenyl]-N¹,N¹-dimethyl-N²-(2-thienylmethyl)alaninamide;
2-{{[3,4-bis(methyloxy)phenyl]methyl}[4-cyano-3-(trifluoromethyl)phenyl]amino}-N,N-dimethylbutanamide;
2-{[4-cyano-3-(trifluoromethyl)phenyl][(3-methyl-2-thienyl)methyl]amino}-N,N-dimethylbutanamide;
2-{[4-cyano-3-(trifluoromethyl)phenyl][(5-methyl-2-furanyl)methyl]amino}-N,N-dimethylbutanamide;
N²-(3-chloro-4-cyanophenyl)-N¹,N¹-dimethyl-N²-(2-phenylethyl)alaninamide;
N²-(3-chloro-4-cyanophenyl)-N²-[2-(2-chlorophenyl)ethyl]-N¹,N¹-dimethylalaninamide;
N²-(3-chloro-4-cyanophenyl)-N¹,N¹-dimethyl-N²-(2-thienylmethyl)norleucinamide;
N²-[4-cyano-3-(trifluoromethyl)phenyl]-N¹,N¹-dimethyl-N²-(2,2,2-trifluoroethyl)norvalinamide;
N²-[4-cyano-3-(trifluoromethyl)phenyl]-N¹,N¹-dimethyl-N²-(2,2,2-trifluoroethyl)norleucinamide;
N²-[4-cyano-3-(trifluoromethyl)phenyl]-N¹,N¹-bis(2-methylpropyl)-N²-(2,2,2-trifluoroethyl)alaninamide;
N¹,N¹-dibutyl-N²-[4-cyano-3-(trifluoromethyl)phenyl]-N²-(2,2,2-trifluoroethyl)alaninamide;
N²-[4-cyano-3-(trifluoromethyl)phenyl]-N¹-(1,1-dimethylethyl)-N¹-methyl-N²-(2,2,2-trifluoroethyl)alaninamide;
N²-[4-cyano-3-(trifluoromethyl)phenyl]-N¹-methyl-N¹-2-propyn-1-yl-N²-(2,2,2-trifluoroethyl)alaninamide;
N²-[4-cyano-3-(trifluoromethyl)phenyl]-N¹-ethyl-N¹-propyl-N²-(2,2,2-trifluoroethyl)alaninamide;
N²-[4-cyano-3-(trifluoromethyl)phenyl]-N¹-ethyl-N¹-(1-methylethyl)-N²-(2,2,2-trifluoroethyl)alaninamide;
N²-[4-cyano-3-(trifluoromethyl)phenyl]-N¹-ethyl-N¹-methyl-N²-(2,2,2-trifluoroethyl)alaninamide;
N²-[4-cyano-3-(trifluoromethyl)phenyl]-N¹,N¹-dipropyl-N²-(2,2,2-trifluoroethyl)alaninamide;
N²-[4-cyano-3-(trifluoromethyl)phenyl]-N¹,N¹-diethyl-N²-(2,2,2-trifluoroethyl)alaninamide;
4-[[1-methyl-2-oxo-2-(1-piperidinyl)ethyl](2,2,2-trifluoroethyl)amino]-2-(trifluoromethyl)benzonitrile;
4-[[2-(1-azetidinyl)-1-methyl-2-oxoethyl](2,2,2-trifluoroethyl)amino]-2-(trifluoromethyl)benzonitrile;
N²-[4-cyano-3-(trifluoromethyl)phenyl]-N¹-methyl-N¹-propyl-N²-(2,2,2-trifluoroethyl)alaninamide;
N¹-butyl-N¹-(cyanomethyl)-N²-[4-cyano-3-(trifluoromethyl)phenyl]-N²-(2,2,2-trifluoroethyl)alaninamide;
N²-[4-cyano-3-(trifluoromethyl)phenyl]-N¹-[2-(methyloxy)ethyl]-N¹-propyl-N²-(2,2,2-trifluoroethyl)alaninamide;
N¹,N¹-bis(cyanomethyl)-N²-[4-cyano-3-(trifluoromethyl)phenyl]-N²-(2,2,2-trifluoroethyl)alaninamide;
4-[[1-methyl-2-(4-morpholinyl)-2-oxoethyl](2,2,2-trifluoroethyl)amino]-2-(trifluoromethyl)benzonitrile;
N²-[4-cyano-3-(trifluoromethyl)phenyl]-N¹-(cyclopropylmethyl)-N¹-propyl-N²-(2,2,2-trifluoroethyl)alaninamide;
N²-[4-cyano-3-(trifluoromethyl)phenyl]-N¹-cyclohexyl-N¹-methyl-N²-(2,2,2-trifluoroethyl)alaninamide;
N²-[4-cyano-3-(trifluoromethyl)phenyl]-N¹-methyl-N¹-[2-(methyloxy)ethyl]-N²-(2,2,2-trifluoroethyl)alaninamide;
4-[[1-methyl-2-oxo-2-(4-thiomorpholinyl)ethyl](2,2,2-trifluoroethyl) amino]-2-(trifluoromethyl)benzonitrile;
N¹-(2-cyanoethyl)-N²-[4-cyano-3-(trifluoromethyl)phenyl]-N¹-methyl-N²-(2,2,2-trifluoroethyl)alaninamide;
4-[[1-methyl-2-oxo-2-(1-pyrrolidinyl)ethyl](2,2,2-trifluoroethyl)amino]-2-(trifluoromethyl)benzonitrile;
N²-[4-cyano-3-(trifluoromethyl)phenyl]-N¹-methyl-N¹-(1-methylethyl)-N²-(2,2,2-trifluoroethyl)alaninamide;
N²-[4-cyano-3-(trifluoromethyl)phenyl]-N¹,N¹-di-2-propen-1-yl-N²-(2,2,2-trifluoroethyl)alaninamide;
N²-[4-cyano-3-(trifluoromethyl)phenyl]-N¹-methyl-N¹-2-propen-1-yl-N²-(2,2,2-trifluoroethyl)alaninamide;
N,N-dibutyl-2-[[4-cyano-3-(trifluoromethyl)phenyl](2,2,2-trifluoroethyl)amino]butanamide;
2-[[4-cyano-3-(trifluoromethyl)phenyl](2,2,2-trifluoroethyl)amino]-N-(1,1-dimethylethyl)-N-methylbutanamide;
2-[[4-cyano-3-(trifluoromethyl)phenyl](2,2,2-trifluoroethyl)amino]-N-methyl-N-2-propyn-1-ylbutanamide;
2-[[4-cyano-3-(trifluoromethyl)phenyl](2,2,2-trifluoroethyl)amino]-N-ethyl-N-propylbutanamide;
2-[[4-cyano-3-(trifluoromethyl)phenyl](2,2,2-trifluoroethyl)amino]-N-ethyl-N-(1-methylethyl)butanamide;
2-[[4-cyano-3-(trifluoromethyl)phenyl](2,2,2-trifluoroethyl)amino]-N,N-bis(2-methylpropyl)butanamide;
2-[[4-cyano-3-(trifluoromethyl)phenyl](2,2,2-trifluoroethyl)amino]-N-ethyl-N-methylbutanamide;
2-[[4-cyano-3-(trifluoromethyl)phenyl](2,2,2-trifluoroethyl)amino]-N,N-dipropyl-butanamide;
2-[[4-cyano-3-(trifluoromethyl)phenyl](2,2,2-trifluoroethyl)amino]-N,N-diethyl-butanamide;
4-[[1-(1-piperidinylcarbonyl)propyl](2,2,2-trifluoroethyl)amino]-2-(trifluoromethyl)benzonitrile;
4-[[1-(1-azetidinylcarbonyl)propyl](2,2,2-trifluoroethyl)amino]-2-(trifluoromethyl)-benzonitrile;
2-[[4-cyano-3-(trifluoromethyl)phenyl](2,2,2-trifluoroethyl)amino]-N-methyl-N-propylbutanamide;
N-butyl-N-(cyanomethyl)-2-[[4-cyano-3-(trifluoromethyl)phenyl](2,2,2-trifluoroethyl)amino]butanamide;
2-[[4-cyano-3-(trifluoromethyl)phenyl](2,2,2-trifluoroethyl)amino]-N-[2-(methyloxy)ethyl]-N-propylbutanamide;
N,N-bis(cyanomethyl)-2-[[4-cyano-3-(trifluoromethyl)phenyl](2,2,2-trifluoroethyl)amino]butanamide;
4-[[1-(4-morpholinylcarbonyl)propyl](2,2,2-trifluoroethyl)amino]-2-(trifluoromethyl)benzonitrile;
2-[[4-cyano-3-(trifluoromethyl)phenyl](2,2,2-trifluoroethyl)amino]-N-(cyclopropylmethyl)-N-propylbutanamide;
2-[[4-cyano-3-(trifluoromethyl)phenyl](2,2,2-trifluoroethyl)amino]-N-cyclohexyl-N-methylbutanamide;
4-[[1-(4-thiomorpholinylcarbonyl)propyl](2,2,2-trifluoroethyl)amino]-2-(trifluoromethyl)benzonitrile;
2-[[4-cyano-3-(trifluoromethyl)phenyl](2,2,2-trifluoroethyl)amino]-N-methyl-N-[2-(methyloxy)ethyl]butanamide;
N-(2-cyanoethyl)-2-[[4-cyano-3-(trifluoromethyl)phenyl](2,2,2-trifluoroethyl)-amino]-N-methylbutanamide;
4-[[1-(1-pyrrolidinylcarbonyl)propyl](2,2,2-trifluoroethyl)amino]-2-(trifluoromethyl)benzonitrile;
2-[[4-cyano-3-(trifluoromethyl)phenyl](2,2,2-trifluoroethyl)amino]-N-methyl-N-(1-methylethyl)butanamide;
2-[[4-cyano-3-(trifluoromethyl)phenyl](2,2,2-trifluoroethyl)amino]-N,N-di-2-propen-1-ylbutanamide;
4-[[1-(1,3-thiazolidin-3-ylcarbonyl)propyl](2,2,2-trifluoroethyl)amino]-2-(trifluoromethyl)benzonitrile;
2-[[4-cyano-3-(trifluoromethyl)phenyl](2,2,2-trifluoroethyl)amino-N-methyl-N-2-propen-1-ylbutanamide; N²-[4-cyano-3-(trifluoromethyl)phenyl]-N¹,N¹-dimethyl-N²-{[2-(trifluoromethyl)-phenyl]methyl}alaninamide;
N²-[4-cyano-3-(trifluoromethyl)phenyl]-N²-(2,2-dimethylpropyl)-N¹,N¹-dimethylalaninamide;
N²-[4-cyano-3-(trifluoromethyl)phenyl]-N²-(3,3-dimethylbutyl)-N¹,N¹-dimethylalaninamide;
N²-[4-cyano-3-(trifluoromethyl)phenyl]-N¹,N¹-dimethyl-N²-(3,3,3-trifluoropropyl)-alaninamide;
N²-[2,2-bis(methyloxy)ethyl]-N²-[4-cyano-3-(trifluoromethyl)phenyl]-N¹,N¹-dimethylalaninamide;
2-[[4-cyano-3-(trifluoromethyl)phenyl](2,2-dimethylpropyl)amino]-N,N-dimethylbutanamide;
N²-[(2chlorophenyl)methyl]-N²-(4-cyanophenyl)-N¹,N¹-dimethylalaninamide;
N²-[4-cyano-3-(trifluoromethyl)phenyl]-N²-(2,2-dimethylpropyl)-N¹,N¹-dimethylnorleucinamide;
N²-(3-chloro-4-cyanophenyl)-N²-ethyl-N¹,N¹-dimethylalaninamide;
N²-(3-chloro-4-cyanophenyl)-N²-[(2-chlorophenyl)methyl]-N¹,N¹-dimethylalaninamide; and
N²-[4-cyano-3-(trifluoromethyl)phenyl]-N²-ethyl-N¹,N¹,2-trimethylalaninamide;
or a pharmaceutically acceptable salt thereof, or a solvate thereof, or a combination thereof.
